(19) United States
(12) Patent Application Publication
(10) Pub. No.: US 2025/0223342 A1
(43) Pub. Date: Jul. 10, 2025

(54) PH-DEPENDENT HSA-BINDING MOLECULES AND METHODS OF USE

(71) Applicant: argenx BV, Ghent (BE)

(72) Inventors: Vladimir Bobkov, Ghent (BE); Karen Silence, Ghent (BE); Jolien Van Santbergen, Ghent (BE); René Bigirimana, Ghent (BE); Judith Baumeister, Ghent (BE); Johannes de Haard, Ghent (BE); Christophe Blanchetot, Ghent (BE)

(21) Appl. No.: 18/979,829

(22) Filed: Dec. 13, 2024

Related U.S. Application Data
(63) Continuation of application No. PCT/EP2023/066179, filed on Jun. 15, 2023.
(60) Provisional application No. 63/352,589, filed on Jun. 15, 2022.

(51) Int. Cl. C07K 16/18; C07K 16/28

(52) U.S. Cl. CPC C07K 16/18; C07K 16/283; C07K 2317/35; C07K 2317/524; C07K 2317/526; C07K 2317/55; C07K 2317/565; C07K 2317/569; C07K 2317/92; C07K 2319/31

(57) ABSTRACT

Provided herein are pH-dependent HSA binding molecules. Fusion proteins comprising pH-dependent HSA binding molecules and a therapeutic protein are also provided. Polynucleotides, vectors, host cells, methods of use, and methods of production are also provided herein.

### CROSS REFERENCE TO RELATED APPLICATIONS

This application is a Continuation of International Patent Application No. PCT/EP2023/066179, filed Jun. 15, 2023, which claims the benefit of and priority to U.S. Provisional Patent Application No. 63/352,589, filed Jun. 15, 2022, the contents of each of which are incorporated herein by reference in their entirety.

### SUBMISSION OF SEQUENCE LISTING XML

The content of the following submission of Sequence Listing XML is incorporated herein by reference in its entirety: a computer readable form (CRF) of the Sequence Listing (file name: P240449WO01_SQL.xml, date created: Jun. 15, 2023, size: 178,913 bytes).

### FIELD

The present disclosure relates to pH-dependent human serum albumin (HSA)-binding molecules and methods of making and using the same.

### BACKGROUND

HSA is the most abundant plasma protein and plays an important role in the binding and transport of common therapeutic agents. HSA is thus an interesting target for exploring and optimizing the pharmacokinetics and pharmacodynamics of a therapeutic agent.

However, considerations need to made to keep the level of HSA in the blood at a healthy level (the normal range is 3.4 to 5.4 g/dL (34 to 54 g/L), as HSA maintains the fluid balance in the body, helps prevent blood vessels from leaking too much, has a role in repairing tissue and helping the body grow while transporting hormones, nutrients and enzymes throughout the body.

Current technologies using HSA-binding to extend half-life of therapeutic agents do so by binding to HSA continuously (e.g., under all physiological conditions), thereby increasing significantly the total size of the therapeutic agent (HSA is ˜66 kDa protein), which can affect efficacy and function by steric hindrance of tissue and tumor penetration and reduced distribution.

Accordingly, there is a need in the art for improved albumin binding molecules that extend the half-life of therapeutic agents without interfering with serum albumin function or therapeutic efficacy.

### SUMMARY

The instant disclosure is broadly directed to HSA-binding molecules that bind preferentially to HSA at acidic pH with reduced or no binding to HSA at physiological pH and fusion proteins thereof.

In an aspect, provided herein is an antigen-binding molecule that specifically binds to HSA, wherein the equilibrium dissociation constant of the antigen-binding molecule to HSA at pH 5.5 and/or pH 6.0 is less than 0.140 mM and the equilibrium dissociation constant of the antigen-binding molecule to HSA at pH 7.4 is at least 2.4 mM.

In some embodiments, the equilibrium dissociation constant of the antigen-binding molecule to HSA at pH 5.5 is less than 0.03 mM and the equilibrium dissociation constant of the antigen-binding molecule to HSA at pH 7.4 is at least 11.5 mM.

In another aspect, provided herein is an antigen-binding molecule that specifically binds to HSA, wherein the binding affinity of the antigen-binding molecule to HSA at pH 5.5 and/or pH 6.0 is at least 10-fold higher than the binding affinity of the antigen-binding molecule to HSA at pH 7.4. In some embodiments, the binding affinity of the HSA-binding molecule to HSA at acidic pH is at least about 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 175-fold, 200-fold, 225-fold, 250-fold, 275-fold, 300-fold, 350-fold, 400-fold, 450-fold, 500-fold, 600-fold, 700-fold, 800-fold, 900-fold, or 1000-fold higher than the binding affinity of the HSA-binding molecule to HSA at physiological pH. In some embodiments, acidic pH is pH 5.5 and/or pH 6.0. In some embodiments, physiological pH is pH 7.4.

In some embodiments, the binding affinity is measured by surface plasmon resonance.

In some embodiments, the antigen-binding molecule is selected from a Fab fragment, a sdAb, an scFv, an antibody mimetic, or an antigen-binding fragment thereof. In some embodiments, the sdAb is a VHH fragment, optionally further comprising one or more amino acids added to the C-terminal end of the VHH fragment. In some embodiments, the one or more amino acids are selected from the group consisting of: A; AG; GG; and PP.

In an aspect, a fusion protein is provided comprising the antigen-binding molecule as described herein and a therapeutic protein.

In some embodiments, the therapeutic protein is a small peptide therapeutic.

In some embodiments, the therapeutic protein is an antibody or fragment thereof, optionally an Fc region or an antigen-binding fragment.

In some embodiments, the antigen-binding molecule is fused to the therapeutic protein via a linker. In some embodiments, the linker is a non-cleavable linker. In some embodiments, the linker is a peptide linker, optionally a GS linker, optionally from 8 to 40 amino acids in length, optionally 20 amino acids in length.

In some embodiments, the antigen-binding molecule is fused to the antibody or fragment thereof via an IgG hinge region or portion thereof.

In some embodiments, the therapeutic protein is an Fc region comprising a first Fc domain and a second Fc domain, and the antigen-binding molecule is fused to the first Fc domain or the second Fc domain via an IgG hinge region or portion thereof.

Also provided is an isolated polynucleotide or polynucleotides encoding any antigen-binding molecule described herein or any fusion protein described herein.

Also provided is an expression vector comprising any isolated polynucleotide or polynucleotides described herein.

Also provided is a host cell comprising any isolated polynucleotide or polynucleotides described herein, or any expression vector described herein.

A method for producing an antigen-binding molecule or a fusion protein is also provided, the method comprising culturing a host cell as described herein under conditions which permit the expression of the antigen-binding molecule or fusion protein.

Also provided is a pharmaceutical composition comprising any antigen-binding molecule described herein or any fusion protein described herein and at least one pharmaceutically acceptable carrier.

Also provided is an antigen-binding molecule as described herein, or a fusion protein as described herein, or a pharmaceutical composition thereof for use as a medicament.

A method of increasing serum half-life of a therapeutic protein is also provided, the method comprising fusing an antigen-binding molecule as described herein to the therapeutic protein.

Also provided is the use of an antigen-binding molecule as described herein to increase serum half-life of a therapeutic protein.

Also provided is the use of any antigen-binding molecule described herein, or any fusion protein described herein, for the manufacture of a medicament.

### BRIEF DESCRIPTION OF THE DRAWINGS

FIG. 1 is a series of sensorgrams showing properties of the selected clones, 2H11 and 11C03, binding to HSA (pH 5.5) on Biacore, as monovalent and bivalent (VHH-Fc) molecules. Association was performed for each run at pH 5.5. Dissociation was performed either at pH 5.5 or 7.4 to investigate pH dependency as indicated.

FIG. 2 is a schematic showing a protocol for study of PK and HSA binding of motavizumab (Mota)-Fab fusion proteins in Albumus mice™.

FIG. 3 is a line graph showing concentration of Mota-Fab fusion proteins in the serum of Albumus mice™ over time.

FIG. 4 is a schematic showing a potential mechanism of endocytic recycling in anti-HSA-VHH with weaker binding of HSA at pH 7.4 and stronger binding at pH 5.5.

### DETAILED DESCRIPTION

The present disclosure provides engineered antigen-binding molecules that specifically bind to HSA (HSA-binding molecules) at acidic pH and do not bind (or bind weakly) to HSA at physiological pH, and fusion proteins thereof. Nucleic acids encoding such HSA-binding molecules or fusion proteins, vectors, host cells, methods of manufacture, and methods for their use are also provided herein.

### Definitions

As used herein, the term “antigen-binding molecule” refers to any polypeptide that specifically binds to an antigen. Examples of antigen-binding domains include polypeptides derived from antibodies, such as Fab fragments, F(ab′)₂ fragments, disulfide-linked Fvs (sdFv), single-chain Fvs (scFv), CDRs, VH domains (VH), VL domains (VL), single-domain antibodies (sdAb), VHH fragments, camelid antibodies, and antigen-binding fragments of any of the above. The term also encompasses synthetic antigen-binding proteins or antibody mimetic proteins such as, for example, anticalins and DARPins.

In some embodiments, the antigen-binding molecule is a VHH fragment. In some embodiments, the VHH fragment has one or more additional amino acids at its C-terminal end. In some embodiments, the one or more additional amino acids are selected from the group consisting of A, AG, GG, and PP.

As used herein, the term “HSA-binding molecule” refers to an antigen-binding molecule that specifically binds to HSA. In some embodiments, HSA comprises an amino acid sequence at least 95% identical to the amino acid sequence provided in GenBank Accession No.: AAA98797.1. In some embodiments, HSA comprises the amino acid sequence provided in GenBank Accession No.: AAA98797.1.

As used herein, the term “affinity” or “binding affinity” refers to the strength of the binding interaction between two molecules. As used herein, the term “equilibrium dissociation constant” or “K_{D}” refers to the propensity of bound complex of two molecules to dissociate into two free molecules. Thus, as the binding affinity increases, the K_{D} decreases.

As used herein, the term “specifically binds” refers to the ability of any molecule to preferentially bind with a given target. For example, a molecule that specifically binds to a given target can bind to other molecules, generally with lower affinity as determined by, e.g., immunoassays, BIAcore™, KinExA 3000 instrument (Sapidyne Instruments, Boise, ID), or other assays known in the art. In a specific embodiment, molecules that specifically bind to a given target bind to the antigen with a K_{D} that is at least 2 logs, 2.5 logs, 3 logs, 4 logs or less than the K_{D} when the molecules bind non-specifically to another target.

As used herein, the term “acidic pH” refers to a pH of less than 7.0, including, but not limited to, a pH of about 5.5, 5.8, 6.0, 6.2, 6.5, or 6.8. In some embodiments, an “acidic pH” refers to an endosomal pH. In some embodiments, an “acidic pH” refers to a pH of from about 5.5 to about 6.0. In some embodiments, an “acidic pH” refers to a pH of from 5.5 to 6.0. In some embodiments, an “acidic pH” refers to a pH of about 5.5 or about 6.0. In some embodiments, an “acidic pH” refers to a pH of 5.5 or 6.0.

As used herein, the term “physiological pH” refers to a pH of 7.2 to 7.6, including, but not limited to, a pH of about 7.2, 7.3, 7.4, 7.5, or 7.6. In some embodiments, a “physiological pH” refers to the pH present in the bloodstream. In some embodiments, a “physiological pH” refers to a pH of from about 7.35 to about 7.45. In some embodiments, a “physiological pH” refers to a pH of from 7.35 to 7.45. In some embodiments, a “physiological pH” refers to a pH of about 7.4. In some embodiments, a “physiological pH” refers to a pH of 7.4.

As used herein, the term “fusion protein” refers to a protein formed by the fusion of at least one antigen-binding molecule described herein to at least one therapeutic protein (or fragment or variant thereof).

As used herein, the term “fused” refers to the linkage of two peptides by a peptide bond or a peptide linker. In some embodiments, two proteins are directly and contiguously fused together by a peptide bond. In some embodiments, two proteins are indirectly and non-contiguously fused through a peptide linker. In some embodiments, one protein is fused to a peptide linker by a peptide bond at a first position, and a second protein is fused to a peptide linker by a peptide bond at a second position.

As used herein, the term “operably linked” refers to a linkage of polynucleotide sequence elements in a functional relationship. For example, a polynucleotide sequence is operably linked when it is placed into a functional relationship with another polynucleotide sequence. In some embodiments, a transcription regulatory polynucleotide sequence, e.g., a promoter, enhancer, or other expression control element is operably linked to a polynucleotide sequence that encodes a protein if it affects the transcription of the polynucleotide sequence that encodes the protein. Operably linked elements may be contiguous or non-contiguous.

As used herein, the term “therapeutic protein” refers to proteins, polypeptides, antibodies, peptides, or fragments or variants thereof, having one or more therapeutic and/or biological activities. Therapeutic proteins include but are not limited to, proteins, polypeptides, peptides, antibodies, and biologics. The terms peptides, proteins, and polypeptides are used interchangeably herein. The term “therapeutic protein” encompasses antibodies and fragments and variants thereof. Thus, a protein disclosed herein may contain at least a fragment or variant of a therapeutic protein, and/or at least a fragment or variant of an antibody. Additionally, the term “therapeutic protein” may refer to the endogenous or naturally-occurring correlate of a therapeutic protein.

By a protein displaying a “therapeutic activity” or a protein that is “therapeutically active,” it is meant that a protein possesses one or more known biological and/or therapeutic activities associated with a therapeutic protein, such as one or more of the therapeutic proteins described herein or otherwise known in the art. As a non-limiting example, a “therapeutic protein” is a protein that is useful to treat, prevent or ameliorate a disease, condition, or disorder. As a non-limiting example, a “therapeutic protein” may be one that binds specifically to a particular cell type (normal (e.g., lymphocytes) or abnormal (e.g., cancer cells)) and therefore may be used to target a compound (drug or cytotoxic agent) to that cell type specifically. Examples of “therapeutic proteins” include, but are not limited to, interferons, enzymes, hormones, growth factors, interleukins, blood clotting factors, antibodies, and fragments and variants thereof.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87:2264-2268, modified as in Karlin S & Altschul S F, (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the terms “antibody” and “antibodies” include full-length antibodies, antigen-binding fragments of full-length antibodies, and molecules comprising antibody CDRs, VH domains (VH), or VL domains (VL). Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single-domain antibodies (sdAb), monovalent antibodies, single chain antibodies or single-chain Fvs (scFv), camelid antibodies, affibody molecules, VHH fragments, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any isotype (e.g., IgG, IgE, IgM, IgD, IgA, or IgY), any subclass (e.g., IgG₁, IgG₂, IgG₃, IgG₄, IgA₁, or IgA₂), or species (e.g., mouse IgG₂ₐ or IgG_{2b}) of immunoglobulin molecule.

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain is an IgG1, IgG2, IgG3, or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype. See, e.g., Jefferis and Lefranc (2009) mAbs 1 (4): 332-338, and de Taeye et al., (2020) Front Immunol. 11:740, incorporated herein by reference in their entirety.

As used herein, the term “variant Fc region” refers to a variant of an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The antigen-binding molecules or fusion proteins of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 1).

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment. In some embodiments, the methods of “treatment” employ administration of a polypeptide to a subject having a disease or disorder, or predisposed to having such a disease or disorder, in order to prevent, cure, delay, reduce the severity of, or ameliorate the disease or disorder or recurring disease or disorder.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the term, “equivalent dose” refers to a dose of a first and a second therapeutic agent wherein the number of molecules of the first and second agents is about the same. In some embodiments, an equivalent dose is an equimolar dose. As used herein, the term “equimolar dose” refers to a dose of a first and a second therapeutic agent wherein the number of moles of the first and second agent is the same. In some embodiments, equivalent dose is calculated using the observed molecular weight of the first and second agents. In some embodiments, equivalent dose is calculated using the predicted molecular weight of the first and second agents. In some embodiments, equivalent dose is calculated using the observed molecular weight of the first agent and the predicted molecular weight of the second agent. In some embodiments, equivalent dose is calculated using the predicted molecular weight of the first agent and the observed molecular weight of the second agent.

As used herein, the terms “pharmacokinetics,” and “PK,” refer to the effect of an organism on a therapeutic agent administered to the organism. In some embodiments, the effect is metabolization and/or clearance of the therapeutic agent. In some embodiments, PK refers to the rate of metabolization and/or clearance of the therapeutic agent. As used herein, the term “improved pharmacokinetics” or “improved PK” refers to the improvement of a desired effect of an organism on a therapeutic agent administered to the organism. In some embodiments, the improved pharmacokinetics includes increase of the half-life (T_{1/2}), clearance, or area under the curve (AUC) of the therapeutic agent in the subject. In some embodiments, the therapeutic agent is a fusion protein or a therapeutic protein as described herein.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of ±20%, ±15%, ±10%, ±5%, ±1%, or ±0.1% of a given value or range, as are appropriate to perform the methods disclosed herein.

As used herein, the term “molecular weight” can refer to a “predicted molecular weight” or an “observed molecular weight.” The “predicted molecular weight” of a protein is a sum of the molecular weights of all the amino acids in the protein. In certain circumstances the “predicted molecular weight” can differ from the “observed molecular weight” of a molecule. In some embodiments, these differences can occur in a protein because of changes in glycosylation, glycanation, ubiquitination, phosphorylation, or protein cleavage of the protein or complexes of additional proteins with a given protein.

### Antigen-Binding Molecules

In an aspect, antigen-binding molecules which specifically bind to HSA (i.e., HSA-binding molecules) are provided by the present disclosure. More specifically, the HSA-binding molecules of the disclosure preferentially bind to HSA at acidic pH with no, or weak, binding to HSA at physiological pH. In some embodiments, the binding affinity of the HSA-binding molecule to HSA at acidic pH is at least about 100-fold higher than the binding affinity of the HSA-binding molecule to HSA at physiological pH. In some embodiments, the binding affinity of the HSA-binding molecule to HSA at acidic pH is at least about 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 175-fold, 200-fold, 225-fold, 250-fold, 275-fold, 300-fold, 350-fold, 400-fold, 450-fold, 500-fold, 600-fold, 700-fold, 800-fold, 900-fold, or 1000-fold higher than the binding affinity of the HSA-binding molecule to HSA at physiological pH. In some embodiments, acidic pH is pH 5.5 and/or pH 6.0. In some embodiments, physiological pH is pH 7.4.

In some embodiments, the equilibrium dissociation constant for binding of the HSA-binding molecule to HSA at acidic pH is less than 0.001, 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.12, 0.13, 0.14, 0.15, 0.16, 0.17, 0.18, 0.19, 0.20, 0.3, 0.4, or 0.5 mM. In some embodiments, the equilibrium dissociation constant for binding of the HSA-binding molecule to HSA at physiological pH is at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8, 5.0, 5.2, 5.4, 5.6, 5.8, 6.0, 6.5, 7.0, 7.5, 8.0, 8.5, 9.0, 9.5, 10.0, 10.5, 11.0, 11.5, 12.0, 13.0, 14.0, or 15.0 mM. In some embodiments, acidic pH is pH 5.5 and/or pH 6.0. In some embodiments, physiological pH is pH 7.4.

In some embodiments, the equilibrium dissociation constant for binding of the HSA-binding molecule to HSA at acidic pH is less than 0.001, 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.12, 0.13, 0.14, 0.15, 0.16, 0.17, 0.18, 0.19, 0.20, 0.3, 0.4, or 0.5 mM and the equilibrium dissociation constant for binding of the HSA-binding molecule to HSA at physiological pH is at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8, 5.0, 5.2, 5.4, 5.6, 5.8, 6.0, 6.5, 7.0, 7.5, 8.0, 8.5, 9.0, 9.5, 10.0, 10.5, 11.0, 11.5, 12.0, 13.0, 14.0, or 15.0 mM. In some embodiments, acidic pH is pH 5.5 and/or pH 6.0. In some embodiments, physiological pH is pH 7.4.

In some embodiments, the equilibrium dissociation constant of the antigen-binding molecule to HSA at pH 5.5 and/or pH 6.0 is less than 0.140 mM and the equilibrium dissociation constant of the antigen-binding molecule to HSA at pH 7.4 is at least 2.4 mM. In some embodiments, the equilibrium dissociation constant of the antigen-binding molecule to HSA at pH 5.5 and/or pH 6.0 is less than 0.03 mM and the equilibrium dissociation constant of the antigen-binding molecule to HSA at pH 7.4 is at least 11.5 mM. In some embodiments, the equilibrium dissociation constant of the antigen-binding molecule to HSA at pH 5.5 and/or pH 6.0 is less than 0.140 mM and the equilibrium dissociation constant of the antigen-binding molecule to HSA at pH 7.4 is at least 11.5 mM. In some embodiments, the equilibrium dissociation constant of the antigen-binding molecule to HSA pH 5.5 and/or pH 6.0 is less than 0.03 mM and the equilibrium dissociation constant of the antigen-binding molecule to HSA at pH 7.4 is at least 2.4 mM.

In some embodiments, the equilibrium dissociation constant of the antigen-binding molecule to HSA at pH 5.5 is less than 0.140 mM and the equilibrium dissociation constant of the antigen-binding molecule to HSA at pH 7.4 is at least 2.4 mM. In some embodiments, the equilibrium dissociation constant of the antigen-binding molecule to HSA at pH 5.5 is less than 0.03 mM and the equilibrium dissociation constant of the antigen-binding molecule to HSA at pH 7.4 is at least 11.5 mM. In some embodiments, the equilibrium dissociation constant of the antigen-binding molecule to HSA at pH 5.5 is less than 0.140 mM and the equilibrium dissociation constant of the antigen-binding molecule to HSA at pH 7.4 is at least 11.5 mM. In some embodiments, the equilibrium dissociation constant of the antigen-binding molecule to HSA pH 5.5 is less than 0.03 mM and the equilibrium dissociation constant of the antigen-binding molecule to HSA at pH 7.4 is at least 2.4 mM.

In some embodiments, the equilibrium dissociation constant of the antigen-binding molecule to HSA at pH 5.5 is less than 0.140 mM and the equilibrium dissociation constant of the antigen-binding molecule to HSA at pH 7.4 is at least 2.4 mM. In some embodiments, the equilibrium dissociation constant of the antigen-binding molecule to HSA at pH 5.5 is less than 0.03 mM and the equilibrium dissociation constant of the antigen-binding molecule to HSA at pH 7.4 is at least 11.5 mM. In some embodiments, the equilibrium dissociation constant of the antigen-binding molecule to HSA at pH 5.5 is less than 0.140 mM and the equilibrium dissociation constant of the antigen-binding molecule to HSA at pH 7.4 is at least 11.5 mM. In some embodiments, the equilibrium dissociation constant of the antigen-binding molecule to HSA pH 5.5 is less than 0.03 mM and the equilibrium dissociation constant of the antigen-binding molecule to HSA at pH 7.4 is at least 2.4 mM.

In some embodiments, the equilibrium dissociation constant of the antigen-binding molecule to HSA at pH 5.5 and/or pH 6.0 is less than 0.03-0.140 mM and the equilibrium dissociation constant of the antigen-binding molecule to HSA at pH 7.4 is at least 2.4-11.5 mM. In some embodiments, the equilibrium dissociation constant of the antigen-binding molecule to HSA at pH 5.5 is less than 0.03-0.140 mM and the equilibrium dissociation constant of the antigen-binding molecule to HSA at pH 7.4 is at least 2.4-11.5 mM.

In some embodiments, at least 75, 76, 77, 78, 79, 80, 81, 82, 83, 84, 85, 86, 87, 88, 89, 90, 91, 92, 93, 94, 95, 96, 97, 98, or 99% of antigen-binding molecule that specifically binds to HSA is bound to HSA at pH 5.5 and/or pH 6.0 and maximum 1, 2, 3, 4, 5, 6, 7, 8, 9, 10, 11, 12, 13, 14, 15, 16, 17, 18, 19, 20, 21, 22, 23, 24 or 25% of the antigen-binding molecule to HSA is free at pH 7.4. In some embodiments, at least 75-99, 80-98, or 85-97% of antigen-binding molecule that specifically binds to HSA is bound to HSA at pH 5.5 and/or pH 6.0 and maximum 1-25, 2-20, or 3-15% of the antigen-binding molecule to HSA is free at pH 7.4.

In some embodiments, at least 80% of antigen-binding molecule that specifically binds to HSA is bound to HSA at pH 5.5 and/or pH 6.0 and maximum 20% of the antigen-binding molecule to HSA is free at pH 7.4. In some embodiments, at least 95% of antigen-binding molecule that specifically binds to HSA is bound to HSA at pH 5.5 and/or pH 6.0 and maximum 5% of the antigen-binding molecule to HSA is free at pH 7.4.

In some embodiments, the HSA-binding molecule does not bind to HSA at physiological pH. In some embodiments, binding of the HSA-binding molecule to HSA is undetectable at physiological pH. In some embodiments, physiological pH is pH 7.4.

Binding affinity of the HSA-binding molecule to HSA can be measured by any known method. In some embodiments, binding affinity of the HSA-binding molecule to HSA is measured by surface plasmon resonance.

In some embodiments, the HSA-binding molecule does not compete with FcRn for HSA binding.

In some embodiments, the antigen-binding molecule is a polypeptide derived from an antibody including, but not limited to, an sdAb (e.g., a VHH fragment), a Fab fragment, an scFv, a VH, or a VL. In some embodiments, the antigen-binding molecule is a synthetic antigen-binding protein or antibody mimetic protein including, but not limited to, an anticalin or a DARPin.

In some embodiments, pH dependent binding of an HSA-binding molecule to HSA may be enhanced by substituting one or more amino acids in one or more CDRs to histidine (H), aspartic acid (D), glutamic acid (E), or alanine (A). In some embodiments, 1, 2, or 3 amino acids in one or more CDRs is independently substituted with H, D, E, or A. In some embodiments, 1, 2, or 3 amino acids in CDR1 is independently substituted with H, D, E, or A. In some embodiments, 1, 2, or 3 amino acids in CDR2 is independently substituted with H, D, E, or A. In some embodiments, 1, 2, or 3 amino acids in CDR3 is independently substituted with H, D, E, or A. In some embodiments, 1, 2, or 3 amino acids in HCDR1 is independently substituted with H, D, E, or A. In some embodiments, 1, 2, or 3 amino acids in HCDR2 is independently substituted with H, D, E, or A. In some embodiments, 1, 2, or 3 amino acids in HCDR3 is independently substituted with H, D, E, or A. In some embodiments, 1, 2, or 3 amino acids in LCDR1 is independently substituted with H, D, E, or A. In some embodiments, 1, 2, or 3 amino acids in LCDR2 is independently substituted with H, D, E, or A. In some embodiments, 1, 2, or 3 amino acids in LCDR3 is independently substituted with H, D, E, or A.

In some embodiments, 1, 2, or 3 amino acids in each of CDR1 and CDR2 is independently substituted with H, D, E, or A. In some embodiments, 1, 2, or 3 amino acids in each of CDR2 and CDR3 is independently substituted with H, D, E, or A. In some embodiments, 1, 2, or 3 amino acids in each of CDR1 and CDR3 is independently substituted with H, D, E, or A. In some embodiments, 1, 2, or 3 amino acids in each of CDR1, CDR2, and CDR3 is independently substituted with H, D, E, or A.

In some embodiments, 1, 2, or 3 amino acids in each of HCDR1 and HCDR2 is independently substituted with H, D, E, or A. In some embodiments, 1, 2, or 3 amino acids in each of HCDR2 and HCDR3 is independently substituted with H, D, E, or A. In some embodiments, 1, 2, or 3 amino acids in each of HCDR1 and HCDR3 is independently substituted with H, D, E, or A. In some embodiments, 1, 2, or 3 amino acids in each of HCDR1, HCDR2, and HCDR3 is independently substituted with H, D, E, or A.

In some embodiments, 1, 2, or 3 amino acids in each of LCDR1 and LCDR2 is independently substituted with H, D, E, or A. In some embodiments, 1, 2, or 3 amino acids in each of LCDR2 and LCDR3 is independently substituted with H, D, E, or A. In some embodiments, 1, 2, or 3 amino acids in each of LCDR1 and LCDR3 is independently substituted with H, D, E, or A. In some embodiments, 1, 2, or 3 amino acids in each of LCDR1, LCDR2, and LCDR3 is independently substituted with H, D, E, or A.

In some embodiments, the antigen-binding molecule further comprises one or more amino acids added at its C-terminus. In some embodiments, the antigen-binding molecule further comprises one or more amino acids added at the C-terminus selected from A, AG, GG, and PP. In some embodiments, the C-terminus of VHH is the amino acid sequence VTVSS (SEQ ID NO: 195). In some embodiments, the C-terminus of VHH consists of the amino acid sequence VTVSS (SEQ ID NO: 195).

In some embodiments, the antigen-binding molecule specifically binds to HSA and is selected from a Fab fragment, an scFv, an sdAb, and antigen-binding fragments thereof. In some embodiments, the antigen-binding domain specifically binds to HSA and is an sdAb, such as a VHH fragment. In some embodiments, HSA comprises an amino acid sequence at least 95% identical to the amino acid sequence provided in GenBank Accession No.: AAA98797.1. In some embodiments HSA comprises the amino acid sequence provided in GenBank Accession No.: AAA98797.1.

In some embodiments, the antigen-binding domain is a VHH fragment comprising CDR1, CDR2, and CDR3 amino acid sequences of a VHH fragment comprising an amino acid sequence selected from SEQ ID NOs: 15-20, 77-88, 90-115, and 186-194.

In some embodiments, the antigen-binding domain is a VHH fragment comprising or consisting of a combination of CDR1, CDR2, and CDR3 wherein 1, 2, 3, 4, or 5 amino acids differ in at least one of the amino acid sequences selected from SEQ ID NOs: 5, 6, and 8; 5, 6, and 9; 5, 6, and 10; 5, 6, and 11; 5, 6, and 12; 5, 6, and 13; 5, 6, and 21; 5, 6, and 22; 5, 6, and 23; 5, 6, and 24; 5, 6, and 25; 5, 6, and 26; 5, 6, and 27; 5, 6, and 28; 5, 6, and 29; 5, 6, and 30; 5, 6, and 31; 5, 6, and 32; 33, 34, and 36; 33, 34, and 37; 33, 34, and 38; 33, 34, and 39; 33, 34, and 40; 33, 34, and 41; 33, 34, and 42; 33, 34, and 43; 33, 34, and 44; 33, 34, and 45; 33, 34, and 46; 33, 34, and 47; 33, 34, and 48; 33, 34, and 49; 33, 34, and 50; 33, 34, and 51; 33, 34, and 52; 33, 34, and 53; 177, 34, and 35; 178, 34, and 35; 33, 179, and 35; 33, 180, and 35; 33, 181, and 35; 33, 182, and 35; 33, 183, and 35; 33, 184, and 35; 33, 185, and 35; 54, 55, and 56; 54, 57, and 58; 59, 60, and 61; 62, 63, and 64; 65, 66, and 67; 68, 69, and 70; 71, 72, and 73; 74, 75, and 76; and 173, 174, and 175.

In some embodiments, the antigen-binding domain is a VHH fragment comprising or consisting of a combination of CDR1, CDR2, and CDR3 selected from SEQ ID NOs: 5, 6, and 8; 5, 6, and 9; 5, 6, and 10; 5, 6, and 11; 5, 6, and 12; 5, 6, and 13; 5, 6, and 21; 5, 6, and 22; 5, 6, and 23; 5, 6, and 24; 5, 6, and 25; 5, 6, and 26; 5, 6, and 27; 5, 6, and 28; 5, 6, and 29; 5, 6, and 30; 5, 6, and 31; 5, 6, and 32; 33, 34, and 36; 33, 34, and 37; 33, 34, and 38; 33, 34, and 39; 33, 34, and 40; 33, 34, and 41; 33, 34, and 42; 33, 34, and 43; 33, 34, and 44; 33, 34, and 45; 33, 34, and 46; 33, 34, and 47; 33, 34, and 48; 33, 34, and 49; 33, 34, and 50; 33, 34, and 51; 33, 34, and 52; 33, 34, and 53; 177, 34, and 35; 178, 34, and 35; 33, 179, and 35; 33, 180, and 35; 33, 181, and 35; 33, 182, and 35; 33, 183, and 35; 33, 184, and 35; 33, 185, and 35; 54, 55, and 56; 54, 57, and 58; 59, 60, and 61; 62, 63, and 64; 65, 66, and 67; 68, 69, and 70; 71, 72, and 73; 74, 75, and 76; and 173, 174, and 175, wherein one or more amino acids within one or more of the CDRs is substituted with an alanine or a histidine.

In some embodiments, the antigen-binding domain is a VHH fragment comprising or consisting of a combination of CDR1, CDR2, and CDR3 selected from SEQ ID NOs: 5, 6, and 8; 5, 6, and 9; 5, 6, and 10; 5, 6, and 11; 5, 6, and 12; 5, 6, and 13; 5, 6, and 21; 5, 6, and 22; 5, 6, and 23; 5, 6, and 24; 5, 6, and 25; 5, 6, and 26; 5, 6, and 27; 5, 6, and 28; 5, 6, and 29; 5, 6, and 30; 5, 6, and 31; 5, 6, and 32; 33, 34, and 36; 33, 34, and 37; 33, 34, and 38; 33, 34, and 39; 33, 34, and 40; 33, 34, and 41; 33, 34, and 42; 33, 34, and 43; 33, 34, and 44; 33, 34, and 45; 33, 34, and 46; 33, 34, and 47; 33, 34, and 48; 33, 34, and 49; 33, 34, and 50; 33, 34, and 51; 33, 34, and 52; 33, 34, and 53; 177, 34, and 35; 178, 34, and 35; 33, 179, and 35; 33, 180, and 35; 33, 181, and 35; 33, 182, and 35; 33, 183, and 35; 33, 184, and 35; 33, 185, and 35; 54, 55, and 56; 54, 57, and 58; 59, 60, and 61; 62, 63, and 64; 65, 66, and 67; 68, 69, and 70; 71, 72, and 73; 74, 75, and 76; and 173, 174, and 175.

In some embodiments, the antigen-binding domain is a VHH fragment comprising or consisting of an amino acid sequence at least 70%, 75%, 80%, 85%, 90%, 95%, 96%, 97%, 98%, or 99% identical to an amino acid sequence selected from SEQ ID NOs: 15-20, 77-88, 90-115, and 186-194. In some embodiments, the antigen-binding domain is a VHH fragment comprising or consisting of an amino acid sequence selected from SEQ ID NOs: 15-20, 77-88, 90-115, and 186-194.

In some embodiments, the antigen-binding domain comprises the amino acid sequence of any one of SEQ ID NOs: 15-20, 77-88, 90-115, and 186-194 or a variant thereof and one or more amino acids added at the C-terminus selected from A, AG, GG, and PP.

In some embodiments, the antigen-binding domain is a Fab or an scFv comprising the HCDR1, HCDR2, and HCDR3 amino acid sequences of a VH comprising the amino acid sequence of SEQ ID NO: 157, 159, 161, 163, 165, 167, 169, or 171 and comprising the LCDR1, LCDR2, and LCDR3 amino acid sequences of a VL comprising the amino acid sequence of SEQ ID NO: 158, 160, 162, 164, 166, 168, 170, or 172.

In some embodiments, the antigen-binding domain is a Fab or an scFv comprising a combination of VHCDR1, VHCDR2, VHCDR3, VLCDR1, VLCDR2, and VLCDR3 of a VH and a VL wherein 1, 2, 3, 4, or 5 amino acids differ in at least one of the amino acid sequences of the CDRs selected from SEQ ID NOs: 157 and 158, 159 and 160, 161 162, 163 and 164, 165 and 166, 167 and 168, 169 and 170, or 171 and 172. In some embodiments, the antigen-binding domain is a Fab or an scFv comprising a combination of VHCDR1, VHCDR2, VHCDR3, VLCDR1, VLCDR2, and VLCDR3 selected from SEQ ID NOs: 157 and 158, 159 and 160, 161 162, 163 and 164, 165 and 166, 167 and 168, 169 and 170, or 171 and 172.

In some embodiments, the antigen-binding domain is a Fab or an scFv consisting of a combination of VHCDR1, VHCDR2, VHCDR3, VLCDR1, VLCDR2, and VLCDR3 of a VH and a VL wherein 1, 2, 3, 4, or 5 amino acids differ in at least one of the amino acid sequences selected from SEQ ID NOs: 157 and 158, 159 and 160, 161 162, 163 and 164, 165 and 166, 167 and 168, 169 and 170, or 171 and 172. In some embodiments, the antigen-binding domain is a Fab or an scFv consisting of a combination of VHCDR1, VHCDR2, VHCDR3, VLCDR1, VLCDR2, and VLCDR3 of a VH and a VL selected from SEQ ID NOs: 157 and 158, 159 and 160, 161 162, 163 and 164, 165 and 166, 167 and 168, 169 and 170, or 171 and 172.

In some embodiments, the antigen-binding domain is a Fab or an scFv comprising or consisting of a combination of HCDR1, HCDR2, and HCDR3 selected from SEQ ID NOs: 116, 117, and 118; 119, 120, and 121; 122, 120, and 121; 123, 124, and 125; 126, 127, and 128; 129, 130, and 131; 132, 133, and 134; and 135, 136, and 137. In some embodiments, the antigen-binding domain is a Fab or an scFv comprising or consisting of a combination of LCDR1, LCDR2, and LCDR3 selected from SEQ ID NOs: 138, 139, and 140; 141, 142, and 143; 144, 145, and 146; 141, 142, and 143; 147, 148, and 149; 150, 151, and 152; 153, 148, and 154; and 155, 151, and 156. In some embodiments, the antigen-binding domain is a Fab or an scFv comprising or consisting of a combination of HCDR1, HCDR2, HCDR3, LCDR1, LCDR2, and LCDR3 selected from SEQ ID NOs: 116, 117, 118, 138, 139, and 140; 119, 120, 121, 141, 142, and 143; 122, 120, 121, 144, 145, and 146; 123, 124, 125, 141, 142, and 143; 126, 127, 128, 147, 148, and 149; 129, 130, 131, 150, 151, and 152; 132, 133, 134, 153, 148, and 154; and 135, 136, 137, 155, 151, and 156.

In some embodiments, the antigen-binding domain is a Fab or an scFv comprising a VH and a VL, wherein the VH comprises an amino acid sequence at least 70%, 75%, 80%, 85%, 90%, 95%, 96%, 97%, 98%, or 99% identical to an amino acid sequence selected from SEQ ID NOs: 157, 159, 161, 163, 165, 167, 169, or 171, and the VL comprises an amino acid sequence at least 70%, 75%, 80%, 85%, 90%, 95%, 96%, 97%, 98%, or 99% identical to an amino acid sequence selected from SEQ ID NOs: 158, 160, 162, 164, 166, 168, 170, or 172. In some embodiments, the antigen-binding domain is a Fab or scFv comprising a VH and a VL, wherein the VH comprises an amino acid sequence selected from SEQ ID NOs: 157, 159, 161, 163, 165, 167, 169, or 171, and wherein the VL comprises an amino acid sequence selected from SEQ ID NOs: 158, 160, 162, 164, 166, 168, 170, or 172.

In some embodiments, the antigen-binding domain is a Fab or an scFv comprising a VH and a VL, wherein the VH and VL comprise amino acid sequences at least 70%, 75%, 80%, 85%, 90%, 95%, 96%, 97%, 98%, or 99% identical to an amino acid sequence selected from SEQ ID NOs: 157 and 158, 159 and 160, 161 162, 163 and 164, 165 and 166, 167 and 168, 169 and 170, or 171 and 172. In some embodiments, the antigen-binding domain is a Fab or an scFv comprising a VH and a VL, wherein the VH and VL comprise the amino acid sequences of SEQ ID NOs: 157 and 158, 159 and 160, 161 162, 163 and 164, 165 and 166, 167 and 168, 169 and 170, or 171 and 172.

### Therapeutic Proteins

A therapeutic protein can be fused to an HSA-binding molecule of the disclosure to extend its half-life. Examples of therapeutic proteins include, but are not limited to, interferons, enzymes, hormones, growth factors, interleukins, blood clotting factors, antibodies, and fragments and variants thereof. In some embodiments, the therapeutic protein is a human protein or is derived from a human protein.

In some embodiments, the therapeutic protein is a therapeutic antibody or fragment thereof.

In some embodiments, the therapeutic protein disclosed herein comprises one or more Fc regions, or fragments thereof, in combination with one or more antigen-binding domains (e.g., a VH, a VL, an sdAb, a Fab fragment, an scFv, or an antibody mimetic). In some embodiments, the therapeutic protein disclosed herein comprises one or more antigen-binding domains (e.g., a VH, a VL, an sdAb, a Fab fragment, an scFv, or an antibody mimetic).

In general, a therapeutic antibody, or fragment thereof, is a human immunoglobulin. It is understood, however, that the therapeutic antibody may be derived from an immunoglobulin of any other mammalian species, including for example, a camelid species, a rodent (e.g., a mouse, rat, rabbit, guinea pig) or non-human primate (e.g., chimpanzee, macaque) species. Moreover, the therapeutic antibody or fragment thereof may be derived from any immunoglobulin class, including IgM, IgG, IgD, IgA, and IgE, and any immunoglobulin isotype, including IgG1, IgG2, IgG3, and IgG4.

To enhance the manufacturability of therapeutic antibodies, and fusion proteins containing the same, as disclosed herein, it is preferable that the constituent Fc regions do not comprise any non-disulfide bonded cysteine residues. Accordingly, in an embodiment, the Fc regions do not comprise a free cysteine residue.

### Fusion Proteins

The disclosure provides fusion proteins comprising an at least one antigen-binding molecule that specifically binds to HSA and at least one therapeutic protein. It has been advantageously found that fusing an HSA-binding molecule of the disclosure to a therapeutic protein increases the half-life of the therapeutic protein due to the ability of the HSA-binding molecule to bind to HSA in the endosome (acidic pH) and thus, be recycled along with HSA. However, since the HSA-binding molecule does not bind, or binds weakly, to HSA at physiological pH in the bloodstream, the fusion protein does not interfere with albumin function. Likewise, the fusion protein retains its efficacy since it is unbound by HSA in the bloodstream.

The therapeutic protein may be any therapeutic protein described herein or otherwise known in the art. The antigen-binding molecule may be any antigen-binding molecule described herein. In some embodiments, the fusion protein comprises only one antigen-binding molecule. In some embodiments, the fusion protein comprises two or more antigen-binding molecules. In some embodiments, the fusion protein comprises only one therapeutic protein. In some embodiments, the fusion protein comprises two or more therapeutic proteins. In some embodiments, the fusion protein comprises one antigen-binding molecule and one therapeutic protein.

In some embodiments, the antigen-binding molecule is fused to the C-terminus of the therapeutic protein. In some embodiments, the antigen-binding molecule is fused to the N-terminus of the therapeutic protein.

In some embodiments, one antigen-binding molecule is fused to the N-terminus of the therapeutic protein and another antigen-binding molecule is fused to the C-terminus of the therapeutic protein. In some embodiments, one antigen-binding molecule is fused to a position other than the N-terminus or the C-terminus of the therapeutic protein and another antigen-binding molecule is fused to the N-terminus of the therapeutic protein. In some embodiments, one antigen-binding molecule is fused to a position other than the N-terminus or the C-terminus of the therapeutic protein and another antigen-binding molecule is fused to the C-terminus of the therapeutic protein.

In some embodiments, one therapeutic protein is fused to the N-terminus of the antigen-binding molecule and another therapeutic protein is fused to the C-terminus of the antigen-binding molecule. In some embodiments, one therapeutic protein is fused to a position other than the N-terminus or the C-terminus of the antigen-binding molecule and another therapeutic protein is fused to the N-terminus of the antigen-binding molecule. In some embodiments, one therapeutic protein is fused to a position other than the N-terminus or the C-terminus of the antigen-binding molecule and another therapeutic protein is fused to the C-terminus of the antigen-binding molecule.

In some embodiments, the therapeutic protein is a therapeutic antibody or a fragment thereof comprising an Fc region. In some embodiments, an antigen-binding molecule is fused to the C-terminus of one or both of the Fc domains of the Fc region. In some embodiments, the antigen-binding molecule is fused to the N-terminus of the one or both of the Fc domains of the Fc region. In some embodiments, the antigen-binding molecule “replaces” one or both of the antigen-binding domains (e.g., a VHH, VH, VL, Fab fragment, scFv, etc.) of the therapeutic antibody. In some embodiments, the antigen-binding molecule is fused to the N-terminus of an antigen-binding domain of the therapeutic antibody.

In some embodiments, one antigen-binding molecule is fused to the C-terminus of one of the Fc domains of the Fc region and another antigen-binding molecule is fused to the C-terminus of the other Fc domain of the Fc region. In some embodiments, one antigen-binding molecule is fused to the N-terminus of one of the Fc domains of the Fc region and another antigen-binding molecule is fused to the N-terminus of the other Fc domain of the Fc region. In some embodiments, one antigen-binding molecule is fused to the N-terminus of one of the Fc domains of the Fc region and another antigen-binding molecule is fused to the C-terminus of the other Fc domain of the Fc region. In some embodiments, one antigen-binding molecule is fused to a position other than the N-terminus or the C-terminus of one of the Fc domains of the Fc region and another antigen-binding molecule is fused to the N-terminus of the other Fc domain of the Fc region. In some embodiments, one antigen-binding molecule is fused to a position other than the N-terminus or the C-terminus of one of the Fc domains of the Fc region and another antigen-binding molecule is fused to the C-terminus of the other Fc domain of the Fc region.

In some embodiments, the antigen-binding molecule may be fused directly to the N-terminus or the C-terminus of a therapeutic protein. In some embodiments, the antigen-binding molecule is fused to the N-terminus or the C-terminus of a therapeutic protein via a linker. In some embodiments, the linker is a non-cleavable linker.

In some embodiments, the antigen-binding molecule may be fused directly to the N-terminus or the C-terminus of an Fc domain. In some embodiments, the antigen-binding molecule is fused to the N-terminus or the C-terminus of an Fc domain via a linker. The linker may be any suitable linker, including those described herein.

### Linkers

The antigen-binding molecule may be fused to the N-terminus or the C-terminus of a therapeutic protein (e.g., an antibody or fragment thereof).

In some embodiments, the antigen-binding molecule may be fused directly to the N-terminus or the C-terminus of a therapeutic protein. In some embodiments, the antigen-binding molecule is linked to the N-terminus or the C-terminus of a therapeutic protein via a linker. In some embodiments, the linker is a non-cleavable linker.

In some embodiments, the antigen-binding molecule may be fused directly to the N-terminus or the C-terminus of an Fc domain. In some embodiments, the antigen-binding molecule is linked to the N-terminus or the C-terminus of an Fc domain via a linker. In some embodiments, the linker is a non-cleavable linker. As used herein, the term “non-cleavable linker” refers to a linker that is not readily cleaved by one or more of a given enzyme, chemical agent, or photo-irradiation. In some embodiments, the enzyme is a protease.

In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis [2-(succinimidooxycarbonyloxy)ethyl] sulfone (BSOCOES), and bis [2-(sulfosuccinimidooxycarbonyloxy)ethyl] sulfone (sulfo-BSOCOES).

Fc domains disclosed herein may comprise a portion of a hinge region. As such, the antigen-binding molecule may be linked to the N-terminus of an Fc domain via this hinge region. In some embodiments, one or more amino acids are included between the C-terminus of the antigen-binding molecule and the N-terminus of the Fc domain. In some embodiments, the one or more amino acids included between the C-terminus of the antigen-binding molecule and the N-terminus of the Fc domain are amino acids of a natural hinge region. In some embodiments, the C-terminus of the antigen-binding molecule is fused to the N-terminus of the Fc domain via a hinge region or a portion thereof. In some embodiments, the hinge region is an IgG hinge region, such as a human IgG hinge region.

In some embodiments, the linker is a peptide linker. Examples of peptide linkers are well known and those of skill in the art could select a suitable peptide linker for use in linking an antigen-binding molecule to a therapeutic protein.

Peptide linkers may be of any length. In some embodiments, the length and amino acid composition of the linker peptide sequence can be optimized to vary the orientation and/or proximity of the antigen-binding molecule and the therapeutic protein to one another to achieve a desired activity of the fusion protein. In some embodiments, the peptide linker is between about 1 and about 100 amino acids in length, between about 8 and about 40 amino acids in length, or between about 15 amino acids and about 25 amino acids in length. In some embodiments, the peptide linker is between 1 and 100 amino acids in length, between 8 and 40 amino acids in length, or between 15 and 25 amino acids in length. In some embodiments, the peptide linker is about 8 amino acid in length, about 9 amino acids in length, about 10 amino acids in length, about 11 amino acids in length, about 12 amino acids in length, about 13 amino acids in length, about 14 amino acids in length, about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24 amino acids in length, about 25 amino acids in length, about 26 amino acids in length, about 27 amino acids in length, about 28 amino acids in length, about 29 amino acids in length, about 30 amino acids in length, about 31 amino acids in length, about 32 amino acids in length, about 33 amino acids in length, about 34 amino acids in length, about 35 amino acids in length, about 36 amino acids in length, about 37 amino acids in length, about 38 amino acids in length, about 39 amino acids in length, or about 40 amino acids in length. In some embodiments, the peptide linker is 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, 30 amino acids in length, 31 amino acids in length, 32 amino acids in length, 33 amino acids in length, 34 amino acids in length, 35 amino acids in length, 36 amino acids in length, 37 amino acids in length, 38 amino acids in length, 39 amino acids in length, or 40 amino acids in length.

In some embodiments, the peptide linker contains only glycine and/or serine residues (e.g., glycine-serine linker or GS linker). Examples of such peptide linkers include: Gly (x) Ser, where x is 0 to 6; or Ser Gly (x), where x is 0 to 6; (Gly Gly Gly Gly Ser)n, wherein n is an integer of one or more; and (Ser Gly Gly Gly Gly)n, wherein n is an integer of one or more. In some embodiments, the peptide linker includes an amino acid sequence selected from the group consisting of: (GGGGS)n and (SGGGG)n, where n is 1 to 8. In some embodiments, the linker peptides are modified such that the amino acid sequence GSG (that occurs at the junction of traditional Gly/Ser linker peptide repeats) is not present. For example, in some embodiments, the peptide linker includes an amino acid sequence selected from the group consisting of: (GGGXX)nGGGGS and GGGGS(XGGGS)n, where X is any amino acid that can be inserted into the sequence and not result in a polypeptide including the sequence GSG, and n is 0 to 4. In some embodiments, the sequence of a linker peptide is (GGGX1X2)nGGGGS and X1 is P and X2 is S and n is 0 to 4. In some other embodiments, the sequence of a linker peptide is (GGGX1X2)nGGGGS and X1 is G and X2 is Q and n is 0 to 4. In some other embodiments, the sequence of a linker peptide is (GGGX1X2)nGGGGS and X1 is G and X2 is A and n is 0 to 4. In yet other embodiments, the sequence of a linker peptide is GGGGS(XGGGS)n, and X is P and n is 0 to 4. In some embodiments, a linker peptide of the disclosure comprises or consists of the amino acid sequence (GGGGA)₂GGGGS. In some embodiments, a linker peptide comprises or consists of the amino acid sequence (GGGGQ)₂GGGGS. In another embodiment, a linker peptide comprises or consists of the amino acid sequence (GGGPS)₂GGGGS. In another embodiment, a linker peptide comprises or consists of the amino acid sequence GGGGS(PGGGS)₂. In yet a further embodiment, a linker peptide comprises or consists of the amino acid sequence GSGGS or SGGSGS. In some embodiments, a linker peptide comprises or consists of the amino acid sequence GGGGSGGGGSGGGGSGGGGS(SEQ ID NO: 2), GGGGSGGGGS(SEQ ID NO: 3), or GGGGSGGGGSGGGGSGGGGSGGGGSGGGGS(SEQ ID NO: 4).

In some embodiments, the peptide linker is a GS linker of about 20 or about 30 amino acids in length. In some embodiments, the peptide linker is a GS linker of 20 or 30 amino acids in length.

### Polynucleotides, Vectors, and Methods of Production

The disclosure also provides polynucleotides encoding the antigen-binding molecules disclosed herein or fragments thereof. In some embodiments, the polynucleotide encodes a therapeutic protein of the disclosure. In some embodiments, the polynucleotide encodes a fusion protein of the disclosure. In some embodiments, the polynucleotide encodes one or more of an antigen-binding molecule, a therapeutic protein, and a linker. In some embodiments, the polynucleotide encodes an antigen-binding molecule and a therapeutic protein, and optionally a linker. In some embodiments, the polynucleotide encodes one or more of an antigen-binding molecule, an Fc region, and a linker. In some embodiments, the polynucleotide encodes an antigen-binding molecule and an Fc region, and optionally a linker. In some embodiments, the polynucleotide encodes a fusion protein comprising one or more antigen-binding molecules and a therapeutic protein. In some embodiments, the polynucleotide encodes one or more of an antigen-binding molecule, an Fc domain, and a linker. In some embodiments, the polynucleotide encodes an antigen-binding molecule and an Fc domain, and optionally a linker. In some embodiments, the polynucleotide encodes a fusion protein comprising one or more antigen-binding molecules and one or more therapeutic proteins.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecules having less than about 15%, 10%, 5%, 2%, 1%, 0.5%, or 0.1% (in particular, less than about 10%) of other material, e.g., cellular material, culture medium, other nucleic acid molecules, chemical precursors and/or other chemicals. In an embodiment, a nucleic acid molecule(s) encoding a polypeptide described herein is isolated or purified.

In an aspect, provided herein are polynucleotides comprising a nucleotide sequence encoding a therapeutic protein or a fusion protein described herein. In another aspect, provided herein are polynucleotides comprising a nucleotide sequence encoding an antigen binding molecule described herein. In another aspect, provided herein are polynucleotides comprising a nucleotide sequence encoding a fusion protein described herein.

The polynucleotides can comprise nucleotide sequences encoding an sdAb (e.g., a VHH fragment), a Fab fragment, an scFv, a VH, or a VL comprising FRs and CDRs of antigen-binding molecules described herein. The polynucleotides can also comprise nucleotide sequences encoding an antibody mimetic as described herein. In some embodiments, the polynucleotides can comprise nucleotide sequences encoding a VHH fragment comprising FR and CDRs of antigen-binding molecules described herein. In some embodiments, the polynucleotides can comprise nucleotide sequences encoding a light chain comprising VL FRs and CDRs of antigen binding molecules described herein or nucleotide sequences encoding a heavy chain comprising VH FRs and CDRs of antigen binding molecules described herein and/or an Fc domain as described herein. In an embodiment, a polynucleotide encodes a VH, VL, heavy chain, and/or light chain of an antigen binding molecules described herein. In an embodiment, a polynucleotide encodes the first VH and the first VL of an antigen binding molecule described herein. In an embodiment, a polynucleotide encodes the second VH and the second VL of an antigen-binding molecule described herein. In an embodiment, a polynucleotide encodes the first heavy chain and the first light chain of an antigen-binding molecule described herein. In an embodiment, a polynucleotide encodes the second heavy chain and the second light chain of an antigen-binding molecule described herein. In an embodiment, a polynucleotide encodes the VH and/or the VL, or the heavy chain and/or the light chain, of an antigen-binding molecule described herein.

In some embodiments, the polynucleotides comprise nucleotide sequences that encode two or more Fc domains. In some embodiments, the polynucleotides comprise nucleotide sequences that encode two Fc domains. In some embodiments, the polynucleotides comprise a first nucleotide sequence that encodes a first Fc domain and a second nucleotide sequence that encodes a second Fc domain. In some embodiments, the first nucleotide sequence and the second nucleotide sequence are comprised in distinct nucleic acid molecules. In some embodiments, the first nucleotide sequence and the second nucleotide sequence are comprised in the same nucleic acid molecule.

In some embodiments, the first and second nucleotide sequence also encode an antigen binding molecule. In some embodiments, the first and second nucleotide sequence encode the same antigen binding molecule. In some embodiments, the first and second nucleotide sequence encode different antigen binding molecules. In some embodiments, the first nucleotide sequence encodes an Fc domain and an antigen binding molecule and the second nucleotide sequence encodes an Fc domain and an antigen-binding domain of a therapeutic antibody. The antigen binding molecules encoded by the first and/or second nucleotide sequences can be any described herein.

In some embodiments, the first and second nucleotide sequence also encode a peptide linker. In some embodiments, the first and second nucleotide sequence encode the same peptide linker. In some embodiments, the first and second nucleotide sequence encode different peptide linkers. In some embodiments, the first nucleotide sequence encodes an Fc domain, a peptide linker, and an antigen binding molecule and the second nucleotide sequence encodes an Fc domain and an antigen-binding domain from a therapeutic antibody. In some embodiments, the first nucleotide sequence encodes an antigen binding molecule, a peptide linker, and an Fc domain and the second nucleotide sequence encodes an Fc domain and an antigen-binding domain from a therapeutic antibody. The peptide linkers encoded by the first and/or second nucleotide sequences can be any described herein. In some embodiments, the linker comprises the amino acid sequence of SEQ ID NO: 2, 3, or 4.

Also provided herein are polynucleotides encoding a polypeptide as provided above that are optimized, e.g., by codon/RNA optimization, replacement with heterologous signal sequences, and elimination of mRNA instability elements. Methods to generate optimized nucleic acids for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid.

The polynucleotides can be obtained, and the nucleotide sequence of the polynucleotides determined, by any method known in the art. Nucleotide sequences encoding proteins described herein, and modified versions of these antibodies can be determined using methods well known in the art, i.e., nucleotide codons known to encode particular amino acids are assembled in such a way to generate a nucleic acid that encodes the protein. Such a polynucleotide encoding the protein can be assembled from chemically synthesized oligonucleotides (e.g., as described in Kutmeier G et al., (1994) BioTechniques 17:242-6, herein incorporated by reference in its entirety), which, briefly, involves the synthesis of overlapping oligonucleotides containing portions of the sequence encoding the antibody, annealing, and ligating of those oligonucleotides, and then amplification of the ligated oligonucleotides by PCR.

Alternatively, a polynucleotide encoding a protein described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the polypeptide of interest. Such PCR amplification methods can be used to obtain nucleic acids comprising the sequence encoding the polypeptide. The amplified nucleic acids can be cloned into vectors for expression in host cells and for further cloning.

If a clone containing a nucleic acid encoding a particular polypeptide is not available, but the sequence of the polypeptide is known, a nucleic acid encoding the polypeptide can be chemically synthesized or obtained from a suitable source (e.g., a cDNA library generated from, or nucleic acid, preferably poly A+ RNA, isolated from any tissue or cells expressing the polypeptide described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide. Amplified nucleic acids generated by PCR can then be cloned into replicable cloning vectors using any method well known in the art.

DNA encoding proteins described herein can be readily isolated and sequenced using conventional procedures. Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce the proteins described herein.

Also provided are polynucleotides that hybridize under high stringency, intermediate or lower stringency hybridization conditions to polynucleotides that encode a protein described herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and has been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In an aspect, provided herein are cells (e.g., host cells) expressing (e.g., recombinantly) a protein described herein, and related polynucleotides and expression vectors. Provided herein are vectors (e.g., expression vectors) comprising polynucleotides comprising nucleotide sequences encoding a protein described herein for recombinant expression in host cells, preferably in mammalian cells (e.g., CHO cells). Also provided herein are host cells comprising such vectors for recombinantly expressing proteins described herein. In an aspect, provided herein are methods for producing a protein described herein, comprising expressing the polypeptide from a host cell.

Recombinant expression of a protein described herein generally involves construction of an expression vector containing a polynucleotide that encodes the polypeptide. Once a polynucleotide encoding a polypeptide described herein has been obtained, the vector for the production of the polypeptide can be produced by recombinant DNA technology using techniques well known in the art. Thus, methods for preparing a protein by expressing a polynucleotide containing a polypeptide encoding nucleotide sequence are described herein. Methods which are well known to those skilled in the art can be used to construct expression vectors containing polypeptide coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing a polypeptide described herein, operably linked to a promoter. Such vectors can, for example, include a nucleotide sequence encoding the constant region of the polypeptide (see, e.g., International Publication Nos. WO 86/05807 and WO 89/01036; and U.S. Pat. No. 5,122,464, which are herein incorporated by reference in their entireties), and variable regions of the polypeptide can be cloned into such a vector for expression of the entire heavy, the entire light chain, or both the entire heavy and light chains.

In an embodiment, a vector comprises a polynucleotide encoding an sdAb, Fab fragment, scFv, VHH fragment, VH, VL, heavy chain, and/or light chain of a polypeptide described herein. In another embodiment, a vector comprises a polynucleotide encoding the VH and the VL of a polypeptide described herein. In another embodiment, a vector comprises a polynucleotide encoding the heavy chain and the light chain of a polypeptide described herein.

An expression vector can be transferred to a cell (e.g., host cell) by conventional techniques and the resulting cells can then be cultured by conventional techniques to produce a polypeptide described herein or a fragment thereof. Thus, provided herein are host cells containing a polynucleotide encoding containing a polypeptide described herein or fragments thereof, or a heavy or light chain thereof, or fragment thereof, or a single chain antibody described herein, operably linked to a promoter for expression of such sequences in the host cell.

In an embodiment, a host cell comprises a polynucleotide comprising one of the first nucleotide sequences and one of the second nucleotide sequences described above. In another embodiment, a host cell comprises a first polynucleotide comprising one of the first nucleotide sequences described above, and a second polynucleotide comprising one of the first nucleotide sequences described above. In another embodiment, a host cell comprises a first vector comprising one of the first nucleotide sequences and one of the second nucleotide sequences described above. In another embodiment, a host cell comprises a first vector comprising one of the first nucleotide sequences and one of the second nucleotide sequences described above, and a second vector comprising a second polynucleotide comprising one of the first nucleotide sequences described above.

In some embodiments, an antigen-binding molecule or fusion protein expressed by a first host cell is associated with an antigen-binding molecule or fusion protein expressed by a second host cell to form a dimer. In some embodiments, provided herein are populations of host cells comprising such first host cells and such second host cells.

In some embodiments, provided herein is a population of vectors comprising a first vector comprising a polynucleotide encoding an antigen-binding molecule or a fusion protein, and a second vector comprising a polynucleotide encoding an antigen-binding molecule or a fusion protein. In some embodiments, provided herein is a population of vectors comprising a first vector comprising a polynucleotide encoding a fusion protein, and a second vector comprising a polynucleotide encoding a therapeutic protein. In some embodiments, provided herein is a population of vectors comprising a first vector comprising a polynucleotide encoding a fusion protein and a second vector comprising a polynucleotide encoding an antigen-binding molecule. In some embodiments, provided herein is a population of vectors comprising a first vector comprising a polynucleotide encoding an antigen-binding molecule and a second vector comprising a polynucleotide encoding a therapeutic protein.

A variety of host-expression vector systems can be utilized to express polypeptides described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express a polypeptide described herein in situ. These include but are not limited to microorganisms such as bacteria (e.g., E. coli and B. subtilis) transformed with, e.g., recombinant bacteriophage DNA, plasmid DNA, or cosmid DNA expression vectors containing protein coding sequences; yeast (e.g., Saccharomyces and Pichia) transformed with, e.g., recombinant yeast expression vectors containing protein coding sequences; insect cell systems infected with, e.g., recombinant virus expression vectors (e.g., baculovirus) containing protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing protein coding sequences; or mammalian cell systems (e.g., COS (e.g., COS1 or COS), CHO, BHK, MDCK, HEK 293, NS0, PER.C6, VERO, CRL7030, HsS78Bst, HeLa, NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing proteins described herein are Chinese hamster ovary (CHO) cells, for example CHO cells from the CHO GS System™ (Lonza). In an embodiment, the heavy chain and/or light chain of an antibody produced by a CHO cell may have an N-terminal glutamine or glutamate residue replaced by pyroglutamate. In an embodiment, cells for expressing polypeptides described herein are human cells, e.g., human cell lines. In an embodiment, a mammalian expression vector is pOptiVEC™ or pcDNA3.3. In an embodiment, bacterial cells such as Escherichia coli, or eukaryotic cells (e.g., mammalian cells), are used for the expression of a recombinant polypeptide. For example, mammalian cells such as CHO cells, in conjunction with a vector such as the major intermediate early gene promoter element from human cytomegalovirus, are an effective expression system for antibodies (Foecking M K & Hofstetter H (1986) Gene 45:101-5; and Cockett M I et al., (1990) Biotechnology 8 (7): 662-7, each of which is herein incorporated by reference in its entirety). In an embodiment, polypeptides described herein are produced by CHO cells or NS0 cells. In an embodiment, the expression of nucleotide sequences encoding polypeptides described herein is regulated by a constitutive promoter, inducible promoter, or tissue specific promoter.

In bacterial systems, a number of expression vectors can be advantageously selected depending upon the use intended for the molecule being expressed. For example, when a large quantity of such a polypeptide is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, HeLa, MDCK, HEK 293, NIH 3T3, W138, BT483, Hs578T, HTB2, BT20, and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells. In an embodiment, proteins described herein are produced in mammalian cells, such as CHO cells.

In an embodiment, a polypeptide described herein comprises a portion of an antibody with reduced fucose content or no fucose content. Such proteins can be produced using techniques known to one skilled in the art. For example, the proteins can be expressed in cells deficient in or lacking the ability to fucosylate. In an example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express a protein described herein can be engineered. In an embodiment, a cell provided herein stably expresses an antigen-binding molecule, a fusion protein, or a therapeutic protein which associate to form polypeptides described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for one to two days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method can advantageously be used to engineer cell lines which express a polypeptide described herein or a fragment thereof. Such engineered cell lines can be particularly useful in the screening and evaluation of compositions that interact directly or indirectly with the polypeptide.

A number of selection systems can be used, including but not limited to the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al., (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of a polypeptide can be increased by vector amplification (for a review, see, Bebbington C R & Hentschel C C G, The use of vectors based on gene amplification for the expression of cloned genes in mammalian cells in DNA cloning, p. 163-188. In DNA Cloning, Vol III, A Practical Approach. D. M. Glover (Ed.) (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the polypeptide will also increase (Crouse G F et al., (1983) Mol Cell Biol 3:257-66, which is herein incorporated by reference in its entirety).

The host cell can be co-transfected with two or more expression vectors described herein. The two vectors can contain identical selectable markers which enable equal expression of polypeptides, such as a first heavy chain and a second heavy chain polypeptide. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both polypeptides. The coding sequences can comprise cDNA or genomic DNA. The expression vector can be monocistronic or multicistronic. A multicistronic nucleic acid construct can encode 2, 3, 4, 5, 6, 7, 8, 9, 10, or more genes/nucleotide sequences, or in the range of 2-5, 5-10, or 10-20 genes/nucleotide sequences. For example, a bicistronic nucleic acid construct can comprise, in the following order, a promoter, a first gene and a second gene. In such an expression vector, the transcription of both genes can be driven by the promoter, whereas the translation of the mRNA from the first gene can be by a cap-dependent scanning mechanism, and the translation of the mRNA from the second gene can be by a cap-independent mechanism, e.g., by an IRES.

Once a polypeptide described herein has been produced by recombinant expression, it can be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins. Further, the polypeptides described herein can be fused to heterologous polypeptide sequences described herein or otherwise known in the art to facilitate purification.

In an embodiment, a polypeptide described herein is isolated or purified. In an embodiment, an isolated polypeptide is one that is substantially free of other polypeptides with different antigenic specificities than the isolated polypeptide. For example, in certain embodiments, a preparation of a protein described herein is substantially free of cellular material and/or chemical precursors. The language “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, 5%, 2%, 1%, 0.5%, or 0.1% (by dry weight) of heterologous protein (also referred to herein as a “contaminating protein”) and/or variants of a polypeptide, for example, different post-translational modified forms of a polypeptide or other different versions of a polypeptide (e.g., polypeptide fragments). When the polypeptide is recombinantly produced, it is also generally substantially free of culture medium, i.e., culture medium represents less than about 20%, 10%, 2%, 1%, 0.5%, or 0.1% of the volume of the protein preparation. When the polypeptide is produced by chemical synthesis, it is generally substantially free of chemical precursors or other chemicals, i.e., it is separated from chemical precursors or other chemicals, which are involved in the synthesis of the protein. Accordingly, such preparations of the protein have less than about 30%, 20%, 10%, or 5% (by dry weight) of chemical precursors or compounds other than the molecule of interest. In an embodiment, polypeptides described herein are isolated or purified.

A polypeptide described herein can be produced by any method known in the art for the synthesis of proteins, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates); Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In an embodiment, a polypeptide described herein is prepared, expressed, created, or isolated by any means that involves creation, e.g., via synthesis, genetic engineering of DNA sequences. In an embodiment, such a polypeptide comprises sequences (e.g., DNA sequences or amino acid sequences) that do not naturally exist within the antibody germline repertoire of an animal or mammal (e.g., human) in vivo.

### Pharmaceutical Compositions

In an aspect, the instant disclosure provides pharmaceutical compositions comprising a fusion protein as disclosed herein for use in methods of treating a disease or disorder. In another aspect, the instant disclosure provides pharmaceutical compositions comprising an antigen-binding molecule as disclosed herein for use in methods of treating a disease or disorder.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., a fusion protein) of the invention (or other prophylactic or therapeutic agent), and a pharmaceutically acceptable carrier.

In some embodiments the pharmaceutical compositions are formulated for administration to a subject via any suitable route of administration including, but not limited to, intramuscular, intravenous, intradermal, intraperitoneal, subcutaneous, epidural, nasal, oral, rectal, topical, inhalation, buccal (e.g., sublingual), and transdermal administration. In an embodiment, the pharmaceutical compositions are formulated to be suitable for intravenous administration to a subject. In an embodiment, the pharmaceutical compositions are formulated to be suitable for subcutaneous administration to a subject.

### Methods

The disclosure provides methods for increasing serum half-life of a therapeutic protein comprising fusing an antigen-binding molecule of the invention to the therapeutic protein. The therapeutic protein may be any therapeutic protein described herein or otherwise known in the art.

In some embodiments, clearance of the fusion protein, comprising an antigen-binding molecule of the invention and a therapeutic protein, is decreased in a subject following a single therapeutic administration of the fusion protein compared to clearance of the therapeutic protein following a single therapeutic administration of the therapeutic protein. In some embodiments, clearance of the fusion protein is decreased by at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, or at least 20-fold in a subject following a single therapeutic administration of the fusion protein compared to clearance of the therapeutic protein following a single therapeutic administration of the therapeutic protein.

In some embodiments, clearance of the fusion protein, comprising an antigen-binding molecule of the invention and a therapeutic protein, is decreased in a subject following a single administration of the fusion protein compared to clearance of the therapeutic protein following a single administration of an equivalent dose of the therapeutic protein. In some embodiments, clearance of the fusion protein is decreased by at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, or at least 20-fold in a subject following a single administration of the fusion protein compared to clearance of the therapeutic protein following a single administration of an equivalent dose of the therapeutic protein.

In some embodiments, terminal half-life (t1/2,z) of the fusion protein, comprising an antigen-binding molecule of the invention and a therapeutic protein, is increased in a subject following a single therapeutic administration of the fusion protein compared to t1/2,z of the therapeutic protein following a single therapeutic administration of the therapeutic protein. In some embodiments, t1/2,z of the fusion protein is increased by at least 0.5-fold, at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, or at least 20-fold in a subject following a single therapeutic administration of the fusion protein compared to t1/2,z of the therapeutic protein following a single therapeutic administration of the therapeutic protein.

In some embodiments, t1/2,z of the fusion protein, comprising an antigen-binding molecule of the invention and a therapeutic protein, is increased in a subject following a single administration of the fusion protein compared to t1/2,z of the therapeutic protein following a single administration of an equivalent dose of the therapeutic protein. In some embodiments, t1/2,z of the fusion protein is increased by at least 0.5-fold, at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, or at least 20-fold in a subject following a single administration of the fusion protein compared to t_{1/2,z} of the therapeutic protein following a single administration of an equivalent dose of the therapeutic protein.

In some embodiments, the efficacy of the fusion protein, comprising an antigen-binding molecule of the invention and a therapeutic protein, is the same, or essentially the same as the efficacy of the therapeutic protein. In some embodiments, the efficacy of the fusion protein is decreased by no more than 10%, 9%, 8%, 7%, 6%, 5%, 4%, 3%, 2%, or 1% compared to the efficacy of the therapeutic protein.

In some embodiments, the efficacy of a dose of a fusion protein, comprising an antigen-binding molecule of the invention and a therapeutic protein, is the same, or essentially the same as the efficacy of an equivalent dose of the therapeutic protein. In some embodiments, the efficacy of a dose of the fusion protein is decreased by no more than 10%, 9%, 8%, 7%, 6%, 5%, 4%, 3%, 2%, or 1% compared to the efficacy of an equivalent dose of the therapeutic protein.

In an embodiment, the fusion protein or antigen-binding molecule does not antagonize FcRn binding to albumin.

In an embodiment, the level of albumin is not decreased in the subject following administration of the fusion protein or antigen-binding molecule compared to a baseline level of albumin. In an embodiment, an albumin reduction of less than about 1%, 2%, 3%, 4%, or 5% compared to baseline albumin level is observed. In an embodiment, an albumin reduction of less than about 10% compared to baseline albumin level is observed.

The disclosure also provides methods for treating a disease or disorder in a subject comprising administering to the subject a therapeutically effective amount of a fusion protein according to the disclosure or a pharmaceutical composition comprising the same.

In an embodiment, the fusion protein is administered to the subject simultaneously or sequentially with an additional therapeutic agent. In an embodiment, the additional therapeutic agent is an anti-inflammatory agent. In an embodiment, the additional therapeutic agent is a corticosteroid. In an embodiment, the additional therapeutic agent is rituximab, daclizumab, basiliximab, muromonab-CD3, infliximab, adalimumab, omalizumab, efalizumab, natalizumab, tocilizumab, eculizumab, golimumab, canakinumab, ustekinumab, or belimumab. In an embodiment, the additional therapeutic agent is a leucocyte depleting agent.

In an embodiment, the additional therapeutic agent is a B-cell depleting agent. In an embodiment, the B-cell depleting agent is an antibody. In an embodiment, the B-cell depleting antibody is an antibody that specifically binds to CD10, CD19, CD20, CD21, CD22, CD23, CD24, CD37, CD53, CD70, CD72, CD74, CD75, CD77, CD79a, CD79b, CD80, CD81, CD82, CD83, CD84, CD85, or CD86.

In some embodiments, the fusion protein is administered intravenously. In some embodiments, the fusion protein is administered intravenously once weekly, once every two weeks, once every three weeks, once every four weeks, once monthly, or once every six weeks.

In some embodiments, the fusion protein is administered subcutaneously. In some embodiments, the fusion protein is administered subcutaneously once weekly, once every two weeks, once every three weeks, once every four weeks, once monthly, or once every six weeks.

### EXAMPLES

The following examples are offered by way of illustration, and not by way of limitation.

### Example 1: Generation of pH Dependent Anti-Albumin VHH/scFV

Briefly, two llamas were immunized with human and phage display libraries were generated (VHH/scFv) using standard procedures. Selection was performed with phage binding to HSA and MSA at pH 5.5 and elution at pH 7.4 (using trypsin as control). Screening of the selected clones was conducted by ELISA and Biacore (human, mouse, and cynomolgus monkey serum albumin) using pH 5.5 or 7.4. Typically, pH dependent clones are selected based on their reduced binding at pH 7.4. Characteristics of select generated clones are shown in Table S1.

TABLE S1
Binding And Sequence Characteristics of Selected VHH clones.

| | Species | DII | Alb23 | K_{D} PH | Kd pH | Kd pH | Ratio |
|---|---|---|---|---|---|---|---|
| Clone | reactivity | binder | competition | 5.5 [nM] | 5.5 [1/s] | 7.4 [1/s] | Kd |
| 2A10 | H, C | No | partially | 96.9 | 3.4E−03 | 1.1E−02 | 3.3 |
| 2C10 | H | No | No | 49.3 | 4.5E−03 | 5.6E−03 | 1.3 |
| 2H11 | H | No | No | 6.84 | 6.3E−04 | 5.2E−02 | 81.9 |
| 9F02 | H, C, M | Yes | Yes | 0.575 | 7.8E−05 | 1.3E−04 | 1.6 |
| 2B03 | H, C | No | partially | 16.6 | 2.9E−03 | 2.5E−02 | 8.7 |
| 1E04 | H, C | No | No | 55.2 | 2.6E−03 | 5.4E−03 | 2.1 |
| 4G12 | H, C | No | No | 12.4 | 1.9E−03 | 3.3E−03 | 1.8 |
| 11C03 | H, C, M | Yes | Yes | 4.12 | 5.1E−04 | 3.9E−03 | 7.7 |
| 9E10 | H, C, M | Yes | Yes | 15.4 | 2.46E−03 | 4.12E−03 | 1.7 |
| 12G03 | H, C, M | No | No | 8.14 | 1.26E−03 | 5.22E−03 | 4.1 |

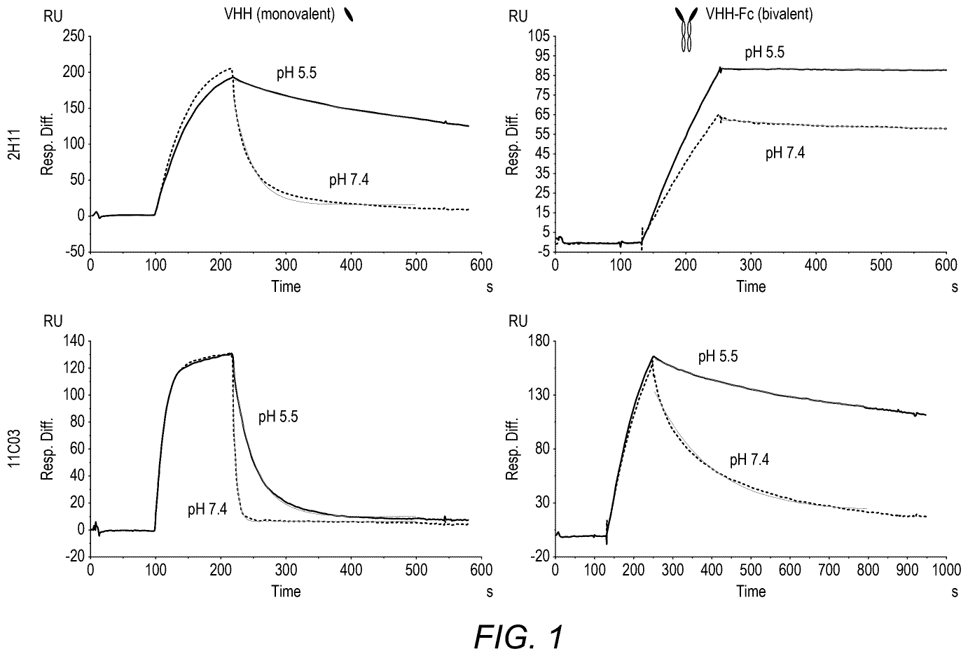

Sensorgrams showing properties of the selected clones, 2H11 and 11C03, binding to HSA (pH 5.5) on Biacore, as monovalent (VHH) and bivalent (VHH-Fc) molecules are shown in FIG. 1. Association was performed for each run at pH 5.5. One clone, 2H11, showed good pH-dependency, no cross-reactivity with mouse and cynomolgus serum albumin, does not bind to isolated DII, and does not compete with Alb23.

In order to have a binding of the VHH to albumin only in the endosome (acidic pH) and very limited (up to none) in circulation (pH 7.4), the affinity of the VHH needs to be adjusted. Indeed, the percentage of complex VHH: Albumin is dependent mainly of the affinity at certain pH. Assuming a concentration of albumin between 34 to 54 mg/mL in circulation and in endosomes, it can be calculated that 80% of the VHH will be not bind (free) if the affinity of the VHH for albumin is around 2.4 mM (calculated for an albumin concentration of 40 mg/ml). Similarly to get at least 80% of the VHH bound to albumin (in the endosome) the affinity must be 0.12 mM (or lower) at pH 5.5. Ideally, less than 5% of the VHH is bound to albumin at pH 7.4 (affinity of 11.5 mM and higher) and more than 95% of the VHH binds albumin at acidic pH (affinity of 30 nM and lower)

To reduce its affinity at pH 7.4, 2H11 was subjected to alanine scanning of all three CDRs. The resulting variant VHHs were produced as two-armed Fc-ABDEG-20GS-Cterm fusions and analyzed by FcRn ELISA QC; Biacore (3000) with human, cynomolgus, and mouse albumin on chip (selection criteria: binding at pH5.5 remains, binding at pH7.4 reduced); and Biacore T200 with Fc-ABDEG-VHH on chip (selection criteria: lowest affinity at pH7.4, highest at pH5.5).

Interestingly, mutations in the CDR3 region of 2H11 increased pH-dependency by reducing binding at pH 7.4 while maintaining good binding at pH 5.5.

Another clone chosen for alanine scanning, 11C03, was cross-reactive with mouse and cynomolgus serum albumin and also showed pH dependent binding. For this clone there were no pronounced effects of alanine mutations in CDR3 on pH-dependency. So binding to HSA was also varied by alanine scanning in the CDR1 and CDR2. A panel of variants displaying reduced binding and different binding at pH 7.4 vs. pH 5.5 was identified on binding to humans (HSA) and cyno albumin (CSA, Table S3).

Data from select 2H11 and 11C03 CDR3 variants are provided in Table S2.

TABLE S2
Analysis of 2H11 and 11C03 CDR3 variants fused at the C-
terminus of Fc-ABDEG with albumin on a chip (Biacore 3000).

| | HSA | | | | |
|---|---|---|---|---|---|
| | Kd pH 5.5, | R0 pH 5.5, | Kd pH 7.4, | R0 pH | R0 |
| Clone | 1/s | RU | 1/s | 7.4, RU | 5.5/7.4 |
| 2H11 | 1.60E−04 | 351 | 7.2E−04 | 115 | 3 |
| parental | | | | | |
| 2H11-v15 | 1.74E−03 | 235 | 2.5E−03 | 0.635 | 370 |
| 2H11-v3 | 1.35E−04 | 164 | 2.9E−03 | 8.24 | 19.9 |
| 2H11-v12 | 9.72E−05 | 93.9 | 1.2E−03 | 6.32 | 14.9 |
| 2H11-v9 | 0.0389* | 77.9 | 1.1E−02* | 0.26 | 300 |
| 2H11-v16 | 3.01E−03 | 8.91 | 1.93E−03 | 0.571 | 16 |
| 2H11-v8 | 1.39E−03 | 1.16 | NA | 0.57 | 2 |
| 11C03 | | 177.21 | | 42.22 | 4.22 |
| parental | | | | | |
| 11C03-v6 | | 39.33 | | 12.08 | 3.26 |

*Difference in off-rate noted.

Data from select 2H11 and 11C03 CDR1-2 variants are provided in Table S3.

TABLE S3
Analysis of 2H11 (CDR3) and 11C03 (CDR1-2) alanine variants fused at the C-terminus
of Fc-ABDEG with albumin on a chip (Biacore 3000) binding to human and Cyno albumin.

| | Human serum albumin | | | | | Cyno serum albumin | | |
|---|---|---|---|---|---|---|---|---|
| | Kd | R0 | Kd pH | R0 | R0 | R0 | R0 | R0 |
| Clone | pH 5.5 | pH 5.5 | 7.4 | pH 7.4 | 5.5/7.4 | pH 5.5 | pH 7.4 | 5.5/7.4 |
| 11C03 parental | | 229 | Z | 57 | 4 | 220 | 33 | 6.7 |
| 11C03 CDR2-V10 | | 136 | | 8.6 | 16 | 105 | 5.65 | 18.6 |
| 11C03 CDR1-V3 | | 109 | | 5.1 | 21 | 103 | 5.2 | 19.8 |
| 11C03 CDR2-V3 | | 68.5 | | 2.4 | 28 | 54.2 | 2.6 | 20.8 |
| 11C03 CDR2-V12 | | 38.7 | | 1.5 | 26 | 26 | 1.35 | 19.3 |
| 11C03 CDR2-V4 | | 12.3 | | 0.826 | 15 | 0 | 2.77 | x |
| 11C03 CDR2-V7 | | 4.57 | | 1.12 | 4 | 0 | 1.09 | x |
| 11C03 CDR1-V2 | | 108 | | 5.82 | 19 | 37.8 | 2.11 | 17.9 |
| 11C03 CDR2-V2 | | 15.1 | | 1.22 | 12 | 5.22 | 1.2 | 4.35 |
| 11C03 CDR2-V5 | | 12.3 | | 1.06 | 12 | 0 | 1.1 | x |
| 2H11 parental | 1.60E-04 | 351 | 7.2E-04 | 115 | 3 | No cross reactivity | | |
| 2H11-v15 | 1.74E-03 | 235 | 2.5E-03 | 0.635 | 370 | to cyno or | | |
| 2H11-v3 | 1.35E-04 | 164 | 2.9E-03 | 8.24 | 19.9 | mouse albumin | | |
| 2H11-v12 | 9.72E-05 | 93.9 | 1.2E-03 | 6.32 | 14.9 | | | |
| 2H11-v9 | 0.0389* | 77.9 | 1.1E-02* | 0.26 | 300 | | | |
| 2H11-v16 | 3.01E-03 | 8.91 | 1.93E-03 | 0.571 | 16 | | | |
| 2H11-v8 | 1.39E-03 | 1.16 | -4.91E-04 | 0.57 | 2 | | | |

*Difference in off-rate noted

Sequences for the 2H11 parent VHH and select variants, 11C03 parent VHH and select variants, additional VHH clones, and scFv clones generated from phage display libraries, as well as Alb23 VHH, are provided below in Tables S4-S8.

TABLE S4
CDR sequences of VHH binding to albumin.

| | | SEQ | | SEQ | | SEQ |
|---|---|---|---|---|---|---|
| | | ID | | ID | | ID |
| Clone | CDR1 | NO. | CDR2 | NO. | CDR3 | NO. |
| 2H11 | SNTMG | 5 | AITWSGGTTYYADSVKG | 6 | EGPKWEPWNGIYHPADFGS | 7 |
| parental | | | | | | |
| 2H11-v9 | SNTMG | 5 | AITWSGGTTYYADSVKG | 6 | EGPKWEPWAGIYHPADFGS | 8 |
| 2H11-v8 | SNTMG | 5 | AITWSGGTTYYADSVKG | 6 | EGPKWEPANGIYHPADFGS | 9 |
| 2H11-v15 | SNTMG | 5 | AITWSGGTTYYADSVKG | 6 | EGPKWEPWNGIYHPAAFGS | 10 |
| 2H11-v3 | SNTMG | 5 | AITWSGGTTYYADSVKG | 6 | EGAKWEPWNGIYHPADFGS | 11 |
| 2H11-v12 | SNTMG | 5 | AITWSGGTTYYADSVKG | 6 | EGPKWEPWNGIAHPADFGS | 12 |
| 2H11-v16 | SNTMG | 5 | AITWSGGTTYYADSVKG | 6 | EGPKWEPWNGIYHPADAGS | 13 |
| 2H11-v1 | SNTMG | 5 | AITWSGGTTYYADSVKG | 6 | AGPKWEPWNGIYHPADFGS | 21 |
| 2H11-v2 | SNTMG | 5 | AITWSGGTTYYADSVKG | 6 | EAPKWEPWNGIYHPADFGS | 22 |
| 2H11-v4 | SNTMG | 5 | AITWSGGTTYYADSVKG | 6 | EGPAWEPWNGIYHPADFGS | 23 |
| 2H11-v5 | SNTMG | 5 | AITWSGGTTYYADSVKG | 6 | EGPKAEPWNGIYHPADFGS | 24 |
| 2H11-v6 | SNTMG | 5 | AITWSGGTTYYADSVKG | 6 | EGPKWAPWNGIYHPADFGS | 25 |
| 2H11-v7 | SNTMG | 5 | AITWSGGTTYYADSVKG | 6 | EGPKWEAWNGIYHPADFGS | 26 |
| 2H11-v10 | SNTMG | 5 | AITWSGGTTYYADSVKG | 6 | EGPKWEPWNAIYHPADFGS | 27 |
| 2H11-v11 | SNTMG | 5 | AITWSGGTTYYADSVKG | 6 | EGPKWEPWNGAYHPADFGS | 28 |
| 2H11-v13 | SNTMG | 5 | AITWSGGTTYYADSVKG | 6 | EGPKWEPWNGIYAPADFGS | 29 |
| 2H11-v14 | SNTMG | 5 | AITWSGGTTYYADSVKG | 6 | EGPKWEPWNGIYHAADFGS | 30 |
| 2H11-v17 | SNTMG | 5 | AITWSGGTTYYADSVKG | 6 | EGPKWEPWNGIYHPADFAS | 31 |
| 2H11-v18 | SNTMG | 5 | AITWSGGTTYYADSVKG | 6 | EGPKWEPWNGIYHPADFGA | 32 |
| 11C03 | AYPMG | 33 | GISWFAGATTDYADSVEG | 34 | SHQLPGGRPGRDMDSYSY | 35 |
| parental | | | | | | |
| 11C03-v1 | AYPMG | 33 | GISWFAGATTDYADSVEG | 34 | AHQLPGGRPGRDMDSYSY | 36 |
| 11C03-v2 | AYPMG | 33 | GISWFAGATTDYADSVEG | 34 | SAQLPGGRPGRDMDSYSY | 37 |
| 11C03-v3 | AYPMG | 33 | GISWFAGATTDYADSVEG | 34 | SHALPGGRPGRDMDSYSY | 38 |
| 11C03-v4 | AYPMG | 33 | GISWFAGATTDYADSVEG | 34 | SHQAPGGRPGRDMDSYSY | 39 |
| 11C03-v5 | AYPMG | 33 | GISWFAGATTDYADSVEG | 34 | SHQLAGGRPGRDMDSYSY | 40 |
| 11C03-v6 | AYPMG | 33 | GISWFAGATTDYADSVEG | 34 | SHQLPAGRPGRDMDSYSY | 41 |
| 11C03-v7 | AYPMG | 33 | GISWFAGATTDYADSVEG | 34 | SHQLPGARPGRDMDSYSY | 42 |
| 11C03-v8 | AYPMG | 33 | GISWFAGATTDYADSVEG | 34 | SHQLPGGAPGRDMDSYSY | 43 |
| 11C03-v9 | AYPMG | 33 | GISWFAGATTDYADSVEG | 34 | SHQLPGGRAGRDMDSYSY | 44 |
| 11C03-v10 | AYPMG | 33 | GISWFAGATTDYADSVEG | 34 | SHQLPGGRPARDMDSYSY | 45 |
| 11C03-v11 | AYPMG | 33 | GISWFAGATTDYADSVEG | 34 | SHQLPGGRPGADMDSYSY | 46 |
| 11C03-v12 | AYPMG | 33 | GISWFAGATTDYADSVEG | 34 | SHQLPGGRPGRAMDSYSY | 47 |
| 11C03-v13 | AYPMG | 33 | GISWFAGATTDYADSVEG | 34 | SHQLPGGRPGRDADSYSY | 48 |
| 11C03-v14 | AYPMG | 33 | GISWFAGATTDYADSVEG | 34 | SHQLPGGRPGRDMASYSY | 49 |
| 11C03-v15 | AYPMG | 33 | GISWFAGATTDYADSVEG | 34 | SHQLPGGRPGRDMDAYSY | 50 |
| 11C03-v16 | AYPMG | 33 | GISWFAGATTDYADSVEG | 34 | SHQLPGGRPGRDMDSASY | 51 |
| 11C03-v17 | AYPMG | 33 | GISWFAGATTDYADSVEG | 34 | SHQLPGGRPGRDMDSYAY | 52 |
| 11C03-v18 | AYPMG | 33 | GISWFAGATTDYADSVEG | 34 | SHQLPGGRPGRDMDSYSA | 53 |
| 11C03- | AAPMG | 177 | GISWFAGATTDYADSVEG | 34 | SHQLPGGRPGRDMDSYSY | 35 |
| CDR1-V2 | | | | | | |
| 11C03- | AYAMG | 178 | GISWFAGATTDYADSVEG | 34 | SHQLPGGRPGRDMDSYSY | 35 |
| CDR1-V3 | | | | | | |
| 11C03- | AYPMG | 33 | GASWFAGATTDYADSVEG | 179 | SHQLPGGRPGRDMDSYSY | 35 |
| CDR2-V2 | | | | | | |
| 11C03- | AYPMG | 33 | GIAWFAGATTDYADSVEG | 180 | SHQLPGGRPGRDMDSYSY | 35 |
| CDR2-V3 | | | | | | |
| 11C03- | AYPMG | 33 | GISAFAGATTDYADSVEG | 181 | SHQLPGGRPGRDMDSYSY | 35 |
| CDR2-V4 | | | | | | |
| 11C03- | AYPMG | 33 | GISWAAGATTDYADSVEG | 182 | SHQLPGGRPGRDMDSYSY | 35 |
| CDR2-V5 | | | | | | |
| 11C03- | AYPMG | 33 | GISWFAAATTDYADSVEG | 183 | SHQLPGGRPGRDMDSYSY | 35 |
| CDR2-V7 | | | | | | |
| 11C03- | AYPMG | 33 | GISWFAGATADYADSVEG | 184 | SHQLPGGRPGRDMDSYSY | 35 |
| CDR2-V10 | | | | | | |
| 11C03- | AYPMG | 33 | GISWFAGATTDAADSVEG | 185 | SHQLPGGRPGRDMDSYSY | 35 |
| CDR2-V12 | | | | | | |
| 2A10 | SYAMG | 54 | TINWSGGSTYYADSVKG | 55 | DLYGPGFVLSMEYDY | 56 |
| 2C10 | SYAMG | 54 | VISWSGGSTYYADSVKG | 57 | EGPKWEPYNGIYSPADFGS | 58 |
| 9F02 | GYAMG | 59 | AISWFAGDTTDYADSVKG | 60 | SSARPMSGRSDEYHY | 61 |
| 2B03 | SYVMA | 62 | SINWSGGSTYYAHSVKG | 63 | DLYGLGYEVMSYDY | 64 |
| 1E04 | NYNAG | 65 | AINWSGGTTYYADSVKG | 66 | DPDWSILNPREYDY | 67 |
| 4G12 | TYGMG | 68 | AINWKGDRYYADSVKG | 69 | DSDGSAFSRRLEYDY | 70 |
| 9E10 | GYAMG | 71 | AISWFAGDTTDYANSVKG | 72 | SSARPLSSRSDEYHY | 73 |
| 12G03 | HSNMA | 74 | HIHWRGSTTYLDSVKG | 75 | DSARRILVTVSTSYDY | 76 |
| Alb23 | SFGMS | 173 | SISGSGSDTLYADSVKG | 174 | GGSLSR | 175 |

TABLE S5
Albumin-binding VHH sequences.

| | | SEQ |
|---|---|---|
| | | ID |
| Clone | VHH | NO. |
| 2H11 | ELQVVESGGGLVQAGGSLRLSCAASGRTFRSNTMGWFRQAPGKEREFVAAITWSGGTTYYADSVK | 14 |
| parental | GRFAISGDNAKNTVYLQMNSLKPEDTAVYYCAAEGPKWEPWNGIYHPADFGSWGQGTQVTVSS | |
| 2H11-v9 | ELQVVESGGGLVQAGGSLRLSCAASGRTFRSNTMGWFRQAPGKEREFVAAITWSGGTTYYADSVK | 15 |
| | GRFAISGDNAKNTVYLQMNSLKPEDTAVYYCAAEGPKWEPWAGIYHPADFGSWGQGTQVTVSS | |
| 2H11-v8 | ELQVVESGGGLVQAGGSLRLSCAASGRTFRSNTMGWFRQAPGKEREFVAAITWSGGTTYYADSVK | 16 |
| | GRFAISGDNAKNTVYLQMNSLKPEDTAVYYCAAEGPKWEPANGIYHPADFGSWGQGTQVTVSS | |
| 2H11-v15 | ELQVVESGGGLVQAGGSLRLSCAASGRTFRSNTMGWFRQAPGKEREFVAAITWSGGTTYYADSVK | 17 |
| | GRFAISGDNAKNTVYLQMNSLKPEDTAVYYCAAEGPKWEPWNGIYHPAAFGSWGQGTQVTVSS | |
| 2H11-v3 | ELQVVESGGGLVQAGGSLRLSCAASGRTFRSNTMGWFRQAPGKEREFVAAITWSGGTTYYADSVK | 18 |
| | GRFAISGDNAKNTVYLQMNSLKPEDTAVYYCAAEGAKWEPWNGIYHPADFGSWGQGTQVTVSS | |
| 2H11-v12 | ELQVVESGGGLVQAGGSLRLSCAASGRTFRSNTMGWFRQAPGKEREFVAAITWSGGTTYYADSVK | 19 |
| | GRFAISGDNAKNTVYLQMNSLKPEDTAVYYCAAEGPKWEPWNGIAHPADFGSWGQGTQVTVSS | |
| 2H11-v16 | ELQVVESGGGLVQAGGSLRLSCAASGRTFRSNTMGWFRQAPGKEREFVAAITWSGGTTYYADSVK | 20 |
| | GRFAISGDNAKNTVYLQMNSLKPEDTAVYYCAAEGPKWEPWNGIYHPADAGSWGQGTQVTVSS | |
| 2H11-v1 | ELQVVESGGGLVQAGGSLRLSCAASGRTFRSNTMGWFRQAPGKEREFVAAITWSGGTTYYADSVK | 77 |
| | GRFAISGDNAKNTVYLQMNSLKPEDTAVYYCAAAGPKWEPWNGIYHPADFGSWGQGTQVTVSS | |
| 2H11-v2 | ELQVVESGGGLVQAGGSLRLSCAASGRTFRSNTMGWFRQAPGKEREFVAAITWSGGTTYYADSVK | 78 |
| | GRFAISGDNAKNTVYLQMNSLKPEDTAVYYCAAEAPKWEPWNGIYHPADFGSWGQGTQVTVSS | |
| 2H11-v4 | ELQVVESGGGLVQAGGSLRLSCAASGRTFRSNTMGWFRQAPGKEREFVAAITWSGGTTYYADSVK | 79 |
| | GRFAISGDNAKNTVYLQMNSLKPEDTAVYYCAAEGPAWEPWNGIYHPADFGSWGQGTQVTVSS | |
| 2H11-v5 | ELQVVESGGGLVQAGGSLRLSCAASGRTFRSNTMGWFRQAPGKEREFVAAITWSGGTTYYADSVK | 80 |
| | GRFAISGDNAKNTVYLQMNSLKPEDTAVYYCAAEGPKAEPWNGIYHPADFGSWGQGTQVTVSS | |
| 2H11-v6 | ELQVVESGGGLVQAGGSLRLSCAASGRTFRSNTMGWFRQAPGKEREFVAAITWSGGTTYYADSVK | 81 |
| | GRFAISGDNAKNTVYLQMNSLKPEDTAVYYCAAEGPKWAPWNGIYHPADFGSWGQGTQVTVSS | |
| 2H11-v7 | ELQVVESGGGLVQAGGSLRLSCAASGRTFRSNTMGWFRQAPGKEREFVAAITWSGGTTYYADSVK | 82 |
| | GRFAISGDNAKNTVYLQMNSLKPEDTAVYYCAAEGPKWEAWNGIYHPADFGSWGQGTQVTVSS | |
| 2H11-v10 | ELQVVESGGGLVQAGGSLRLSCAASGRTFRSNTMGWFRQAPGKEREFVAAITWSGGTTYYADSVK | 83 |
| | GRFAISGDNAKNTVYLQMNSLKPEDTAVYYCAAEGPKWEPWNAIYHPADFGSWGQGTQVTVSS | |
| 2H11-v11 | ELQVVESGGGLVQAGGSLRLSCAASGRTFRSNTMGWFRQAPGKEREFVAAITWSGGTTYYADSVK | 84 |
| | GRFAISGDNAKNTVYLQMNSLKPEDTAVYYCAAEGPKWEPWNGAYHPADFGSWGQGTQVTVSS | |
| 2H11-v13 | ELQVVESGGGLVQAGGSLRLSCAASGRTFRSNTMGWFRQAPGKEREFVAAITWSGGTTYYADSVK | 85 |
| | GRFAISGDNAKNTVYLQMNSLKPEDTAVYYCAAEGPKWEPWNGIYAPADFGSWGQGTQVTVSS | |
| 2H11-v14 | ELQVVESGGGLVQAGGSLRLSCAASGRTFRSNTMGWFRQAPGKEREFVAAITWSGGTTYYADSVK | 86 |
| | GRFAISGDNAKNTVYLQMNSLKPEDTAVYYCAAEGPKWEPWNGIYHAADFGSWGQGTQVTVSS | |
| 2H11-v17 | ELQVVESGGGLVQAGGSLRLSCAASGRTFRSNTMGWFRQAPGKEREFVAAITWSGGTTYYADSVK | 87 |
| | GRFAISGDNAKNTVYLQMNSLKPEDTAVYYCAAEGPKWEPWNGIYHPADFASWGQGTQVTVSS | |
| 2H11-v18 | ELQVVESGGGLVQAGGSLRLSCAASGRTFRSNTMGWFRQAPGKEREFVAAITWSGGTTYYADSVK | 88 |
| | GRFAISGDNAKNTVYLQMNSLKPEDTAVYYCAAEGPKWEPWNGIYHPADFGAWGQGTQVTVSS | |
| 11C03 | QLQVVESGGGLVQAGGSLRLSCAASERTFVAYPMGWFRQAPEKEREFVAGISWFAGATTDYADSV | 89 |
| parental | EGRFTISRDNAKNTAYLQMNNLKPEDTAVYYCAISHQLPGGRPGRDMDSYSYWGHGTQVTVSS | |
| 11C03-v1 | QLQVVESGGGLVQAGGSLRLSCAASERTFVAYPMGWFRQAPEKEREFVAGISWFAGATTDYADSV | 90 |
| | EGRFTISRDNAKNTAYLQMNNLKPEDTAVYYCAIAHQLPGGRPGRDMDSYSYWGHGTQVTVSS | |
| 11C03-v2 | QLQVVESGGGLVQAGGSLRLSCAASERTFVAYPMGWFRQAPEKEREFVAGISWFAGATTDYADSV | 91 |
| | EGRFTISRDNAKNTAYLQMNNLKPEDTAVYYCAISAQLPGGRPGRDMDSYSYWGHGTQVTVSS | |
| 11C03-v3 | QLQVVESGGGLVQAGGSLRLSCAASERTFVAYPMGWFRQAPEKEREFVAGISWFAGATTDYADSV | 92 |
| | EGRFTISRDNAKNTAYLQMNNLKPEDTAVYYCAISHALPGGRPGRDMDSYSYWGHGTQVTVSS | |
| 11C03-v4 | QLQVVESGGGLVQAGGSLRLSCAASERTFVAYPMGWFRQAPEKEREFVAGISWFAGATTDYADSV | 93 |
| | EGRFTISRDNAKNTAYLQMNNLKPEDTAVYYCAISHQAPGGRPGRDMDSYSYWGHGTQVTVSS | |
| 11C03-v5 | QLQVVESGGGLVQAGGSLRLSCAASERTFVAYPMGWFRQAPEKEREFVAGISWFAGATTDYADSV | 94 |
| | EGRFTISRDNAKNTAYLQMNNLKPEDTAVYYCAISHQLAGGRPGRDMDSYSYWGHGTQVTVSS | |
| 11C03-v6 | QLQVVESGGGLVQAGGSLRLSCAASERTFVAYPMGWFRQAPEKEREFVAGISWFAGATTDYADSV | 95 |
| | EGRFTISRDNAKNTAYLQMNNLKPEDTAVYYCAISHQLPAGRPGRDMDSYSYWGHGTQVTVSS | |
| 11C03-v7 | QLQVVESGGGLVQAGGSLRLSCAASERTFVAYPMGWFRQAPEKEREFVAGISWFAGATTDYADSV | 96 |
| | EGRFTISRDNAKNTAYLQMNNLKPEDTAVYYCAISHQLPGARPGRDMDSYSYWGHGTQVTVSS | |
| 11C03-v8 | QLQVVESGGGLVQAGGSLRLSCAASERTFVAYPMGWFRQAPEKEREFVAGISWFAGATTDYADSV | 97 |
| | EGRFTISRDNAKNTAYLQMNNLKPEDTAVYYCAISHQLPGGAPGRDMDSYSYWGHGTQVTVSS | |
| 11C03-v9 | QLQVVESGGGLVQAGGSLRLSCAASERTFVAYPMGWFRQAPEKEREFVAGISWFAGATTDYADSV | 98 |
| | EGRFTISRDNAKNTAYLQMNNLKPEDTAVYYCAISHQLPGGRAGRDMDSYSYWGHGTQVTVSS | |
| 11C03-v10 | QLQVVESGGGLVQAGGSLRLSCAASERTFVAYPMGWFRQAPEKEREFVAGISWFAGATTDYADSV | 99 |
| | EGRFTISRDNAKNTAYLQMNNLKPEDTAVYYCAISHQLPGGRPARDMDSYSYWGHGTQVTVSS | |
| 11C03-v11 | QLQVVESGGGLVQAGGSLRLSCAASERTFVAYPMGWFRQAPEKEREFVAGISWFAGATTDYADSV | 100 |
| | EGRFTISRDNAKNTAYLQMNNLKPEDTAVYYCAISHQLPGGRPGADMDSYSYWGHGTQVTVSS | |
| 11C03-v12 | QLQVVESGGGLVQAGGSLRLSCAASERTFVAYPMGWFRQAPEKEREFVAGISWFAGATTDYADSV | 101 |
| | EGRFTISRDNAKNTAYLQMNNLKPEDTAVYYCAISHQLPGGRPGRAMDSYSYWGHGTQVTVSS | |
| 11C03-v13 | QLQVVESGGGLVQAGGSLRLSCAASERTFVAYPMGWFRQAPEKEREFVAGISWFAGATTDYADSV | 102 |
| | EGRFTISRDNAKNTAYLQMNNLKPEDTAVYYCAISHQLPGGRPGRDADSYSYWGHGTQVTVSS | |
| 11C03-v14 | QLQVVESGGGLVQAGGSLRLSCAASERTFVAYPMGWFRQAPEKEREFVAGISWFAGATTDYADSV | 103 |
| | EGRFTISRDNAKNTAYLQMNNLKPEDTAVYYCAISHQLPGGRPGRDMASYSYWGHGTQVTVSS | |
| 11C03-v15 | QLQVVESGGGLVQAGGSLRLSCAASERTFVAYPMGWFRQAPEKEREFVAGISWFAGATTDYADSV | 104 |
| | EGRFTISRDNAKNTAYLQMNNLKPEDTAVYYCAISHQLPGGRPGRDMDAYSYWGHGTQVTVSS | |
| 11C03-v16 | QLQVVESGGGLVQAGGSLRLSCAASERTFVAYPMGWFRQAPEKEREFVAGISWFAGATTDYADSV | 105 |
| | EGRFTISRDNAKNTAYLQMNNLKPEDTAVYYCAISHQLPGGRPGRDMDSASYWGHGTQVTVSS | |
| 11C03-v17 | QLQVVESGGGLVQAGGSLRLSCAASERTFVAYPMGWFRQAPEKEREFVAGISWFAGATTDYADSV | 106 |
| | EGRFTISRDNAKNTAYLQMNNLKPEDTAVYYCAISHQLPGGRPGRDMDSYAYWGHGTQVTVSS | |
| 11C03-v18 | QLQVVESGGGLVQAGGSLRLSCAASERTFVAYPMGWFRQAPEKEREFVAGISWFAGATTDYADSV | 107 |
| | EGRFTISRDNAKNTAYLQMNNLKPEDTAVYYCAISHQLPGGRPGRDMDSYSAWGHGTQVTVSS | |
| 11C03- | QLQVVESGGGLVQAGGSLRLSCAASERTFVAAPMGWFRQAPEKEREFVAGISWFAGATTDYADSV | 186 |
| CDR1-V2 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYAMGWFRQAPEKEREFVAGISWFAGATTDYADSV | 187 |
| CDR1-V3 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGASWFAGATTDYADS | 188 |
| CDR2-V2 | VEGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGIAWFAGATTDYADSV | 189 |
| CDR2-V3 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGISAFAGATTDYADSV | 190 |
| CDR2-V4 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGISWAAGATTDYADSV | 191 |
| CDR2-V5 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGISWFAAATTDYADSV | 192 |
| CDR2-V7 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGISWFAGATADYADSV | 193 |
| CDR2-V10 | EGRFTISRDNAKNTAYLQMNNLKPEDTAVYYCAISHQLPGGRPGRDMDSYSYWGHGTQVTVSS | |
| 11C03- | QLQVVESGGGLVQAGGSLRLSCAASERTFVAYPMGWFRQAPEKEREFVAGISWFAGATTDAADSV | 194 |
| CDR2-V12 | EGRFTISRDNAKNTAYLQMNNLKPEDTAVYYCAISHQLPGGRPGRDMDSYSYWGHGTQVTVSS | |
| 2A10 | EVQLVESGGGLVQAGGSLRLSCAASGRTFSSYAMGWFRQAPGKEREFVATINWSGGSTYYADSVK | 108 |
| | GRFTISRDNAKNTVYLQMNSLKPEDTAVYYCAADLYGPGFVLSMEYDYWGQGTQVTVSS | |
| 2C10 | QLQLVESGGGLVQAGGSLRLSCAASGRTFSSYAMGWFRQAPGKEREFVAVISWSGGSTYYADSVK | 109 |
| | GRFTISGDNAKNTVYLQMNSLKPEDTAVYYCATEGPKWEPYNGIYSPADFGSWGQGTQVTVSS | |
| 9F02 | EVQLVESGGGLVQAGGSLRLSCTASRRAFIGYAMGWFRQAPGKEREFVAAISWFAGDTTDYADSV | 110 |
| | KGRFTISRDNAKNTVYLQMNGLKPEDTAVYVCAASSARPMSGRSDEYHYWGQGTQVTVSS | |
| 2B03 | QLQLVESGGGLVQAGGSLRLSCAASGRAISSYVMAWFRQAPGKEREFVTSINWSGGSTYYAHSVK | 111 |
| | GRFTISRDNAKNTVYLQMNTLKPEDTAVYYCAADLYGLGYEVMSYDYWGQGTQVTVSS | |
| 1E04 | QLQLVESGGGLVQTGGSLRLSCAASGRTFSNYNAGWFRQVPGKEPEFVAAINWSGGTTYYADSVK | 112 |
| | GRFTISRDNAKNTVYLQMNSLKPEDTAVYYCAADPDWSILNPREYDYWGQGTRVTVSS | |
| 4G12 | QLQLVESGGGLVQAGGSLRLSCVASGRSFSTYGMGWFRQAPGAEREFAAAINWKGDRYYADSVK | 113 |
| | GRFTISRDNAKNTVFLEMNSLKPEDTGVYYCAADSDGSAFSRRLEYDYWGQGTQVTVSS | |
| 9E10 | QLQVVESGGGLVQAGGSLRLSCTTSGRAFIGYAMGWFRQAPGKEREFVAAISWFAGDTTDYANSV | 114 |
| | KGRFTISRDNAKNTVYLQMNSLKPEDTAVYVCAASSARPLSSRSDEYHYWGQGTQVTVSS | |
| 12G03 | QVQLVESGGGLVQAGGSLRLSCAASERAFSHSNMAWFRQAPGKEREFVAHIHWRGSTTYLDSVKG | 115 |
| | RFTISRDNAKNTVYLQMNGLRLEDTAVYYCAADSARRILVTVSTSYDYWGQGTQVTVSS | |
| Alb23 | EVQLLESGGGLVQPGGSLRLSCAASGFTFRSFGMSWVRQAPGKGPEWVSSISGSGSDTLYADSVKG | 176 |
| | RFTISRDNSKNTLYLQMNSLRPEDTAVYYCTIGGSLSRSSQGTLVTVSS | |

TABLE S6
CDR sequences of VH binding to albumin.

| | | SEQ | | SEQ | | SEQ |
|---|---|---|---|---|---|---|
| | | ID | | ID | | ID |
| Clone | CDR1 | NO. | CDR2 | NO. | CDR3 | NO. |
| 267 | SYWMY | 116 | VINTSGGSTYYADSVKG | 117 | GGLVVAGGLGS | 118 |
| 405 | MYWMY | 119 | AIDTRGGSTYYADSVKG | 120 | GGLVRVGGLGS | 121 |
| 408 | YYWMY | 122 | AIDTRGGSTYYADSVKG | 120 | GGLVRVGGLGS | 121 |
| 415 | HYYMN | 123 | GINTGGGTTYYADSVKG | 124 | GGIVAVGGFGS | 125 |
| 438 | RYWIH | 126 | GISIGDGTTYYADSVRG | 127 | GGIVYFGAYDS | 128 |
| 442 | DFVMH | 129 | AIRWDDGSTYYAESMKG | 130 | SVSASSNY | 131 |
| 462 | RYWMH | 132 | GINTGTGSTAYADSVKG | 133 | GGLVVGGGYSD | 134 |
| 476 | RYWIH | 135 | GISIGDGTTYYADSVRG | 136 | GGIVYFGAYDS | 137 |

TABLE S7
CDR sequences of VL binding to albumin.

| | | SEQ | | SEQ | | SEQ |
|---|---|---|---|---|---|---|
| | | ID | | ID | | ID |
| Clone | CDR1 | NO. | CDR2 | NO. | CDR3 | NO. |
| 267 | TGSSSNIGDNYVN | 138 | DNTKRAS | 139 | SSWDDSLSGLV | 140 |
| 405 | GGDNIGSKNAH | 141 | KDSNRPS | 142 | QVWDSSANAAV | 143 |
| 408 | AGTSSDVGYGNYVS | 144 | DVSKRAS | 145 | ASYRSGTTV | 146 |
| 415 | GGDNIGSKNAH | 141 | KDSNRPS | 142 | QVWDSSANAAV | 143 |
| 438 | KSSQSVVSGSNQKS | 147 | LASTRES | 148 | QQGYGTPFS | 149 |
| | LLN | | | | | |
| 442 | QASQNIISYLA | 150 | GASRLQT | 151 | LQHLTYPLT | 152 |
| 462 | KSSRSVLYSSNQKN | 153 | LASTRES | 148 | QQGYDVPVT | 154 |
| | YLA | | | | | |
| 476 | QASQSISSYLA | 155 | GASRLQT | 151 | LQQKLPVT | 156 |

TABLE S8
Albumin-binding VH/VL sequences.

| | | SEQ |
|---|---|---|
| | | ID |
| Clone | VH/VL | NO. |
| 267 | EVQLVESGGGLVQPGGSLRLSCAASGFTFSSYW | 157 |
| VH | MYWVRQAPGKGLEWVSVINTSGGSTYYADSVKG | |
| | RFTISRDNAKNTLYLQMNNLKSEDTAVYYCAKG | |
| | GLVVAGGLGSWGQGTQVTVSS | |
| 267 | QPVLNQPPSVSGSPGQKFTISCTGSSSNIGDNY | 158 |
| VL | VNWYQHLPGTAPKLLIYDNTKRASGVPDRFSGS | |
| | KSGTSASLTITELQAEDEADYYCSSWDDSLSGL | |
| | VFGGGTRLTVL | |
| 405 | EVQLQESGGGLVQPGGSLRLSCAASGFTFSMYW | 159 |
| VH | MYWVRQAPGKGLEWVSAIDTRGGSTYYADSVKG | |
| | RFTISRDNAKNTLYLQMNSLKPEDTALYYCATG | |
| | GLVRVGGLGSWGQGTQVTVSS | |
| 405 | SYELTQSPSVSVALRQTAKITCGGDNIGSKNAH | 160 |
| VL | WYQQKPGQAPVLVIYKDSNRPSGIPERFSGSNS | |
| | GNTATLTVSGAQAEDEADYYCQVWDSSANAAVF | |
| | GGGTHLTVL | |
| 408 | QVQLVESGGGLVQPGGSLRLSCAASGFTFSYYW | 161 |
| VH | MYWVRQAPGKGLEWVSAIDTRGGSTYYADSVKG | |
| | RFTISRDNAKNTLYLQMNSLKPEDTALYYCARG | |
| | GLVRVGGLGSWGQGTQVTVSS | |
| 408 | QAVLTQPPSVSGSPGKTVTISCAGTSSDVGYGN | 162 |
| VL | YVSWYQQLPGMAPKLLIYDVSKRASGITDRFSA | |
| | SKSGNTASLTISGLQSEDEADYYCASYRSGTTV | |
| | FGGGTHLTVL | |
| 415 | ELQLVESGGGLVQPGGSLRVSCAASGFTFSHYY | 163 |
| VH | MNWVRQAPGKGLEWVSGINTGGGTTYYADSVKG | |
| | RFTISRDNAKNTLYLQMNSLKPEDTALYYCAKG | |
| | GIVAVGGFGSWGQGTQVTVSS | |
| 415 | SYELTQSPSVSVALRQTAKITCGGDNIGSKNAH | 164 |
| VL | WYQQKPGQAPVLVIYKDSNRPSGIPERFSGSNS | |
| | GNTATLTVSGAQAEDEADYYCQVWDSSANAAVF | |
| | GGGTHLTVL | |
| 438 | EVQLQESGGGLVQPGGSLRLSCAASGFTFGRYW | 165 |
| VH | IHWVRQAPGKGLEWVSGISIGDGTTYYADSVRG | |
| | RFTISRDKAKNTLALTMNSLKSEDTAVYYCAKG | |
| | GIVYFGAYDSWGQGTQVTVSS | |
| 438 | DIVMTQSPRSVTASVGEKVTINCKSSQSVVSGS | 166 |
| VL | NQKSLLNWYQQRPGQSPRLLIYLASTRESGVPD | |
| | RFSGSGSTTDFTLTISSFQPEDVAVYYCQQGYG | |
| | TPFSFGSGTRLEIK | |
| 442 | ELQLVESGGGLVQPGGSLRLSCAASGFTSDDFV | 167 |
| VH | MHWVRQAPGKGLEWVSAIRWDDGSTYYAESMKG | |
| | RFTISRDNAKNTLYLQMNSLKSEDTAVYYCAKS | |
| | VSASSNYWGQGTQVTVSS | |
| 442 | DIVMTQSPSSLSVSLGDRVTITCQASQNIISYL | 168 |
| VL | AWYQQKPGQTPKLLIYGASRLQTGVPSRFSGSG | |
| | SGTSFTLTISGVEAEDVATYYCLQHLTYPLTFG | |
| | QGTKVELK | |
| 462 | ELQLVESGGGLVQPGGSLRLACAASGFTFSRYW | 169 |
| VH | MHWVRQAPGKGLEWVSGINTGTGSTAYADSVKG | |
| | RFTISRDNGKNTLYLQMNSLKSDDTAVYYCAKG | |
| | GLVVGGGYSDWGQGTQVTVSS | |
| 462 | DIVMTQSPSSVTASAGEKVTINCKSSRSVLYSS | 170 |
| VL | NQKNYLAWYQQRPGQSPRLLIYLASTRESGVPD | |
| | RFSGSGSTTDFTLTISSFQPEDVAVYYCQQGYD | |
| | VPVTFGQGTKVELK | |
| 476 | QVQVQESGGGLVQPGGSLRLSCAASGFTFGRYW | 171 |
| VH | IHWVRQAPGKGLEWVSGISIGDGTTYYADSVRG | |
| | RFTISRDKAKNTLALTMNSLKSEDTAVYYCAKG | |
| | GIVYFGAYDSWGQGTQVTVSS | |
| 476 | DIVMTQSPSSLSASLGDRVTITCQASQSISSYL | 172 |
| VL | AWYQQKPGQAPKLLIYGASRLQTGVPSRFSGGG | |
| | SGTSFTLTISGVEAEDVATYYCLQQKLPVTFGQ | |
| | GTKVELK | |

### Example 2: Evaluation of pH-Dependent Anti-Albumin VHHs to Extend Half-Life of Fab Fragments in Albumus Mice™ Independently of IgG-FcRn Mediated Recycling

The affinity requirements to prolong half-life with anti-albumin VHHs with different degrees of pH dependency (stronger binding at pH 5.5, weaker binding at pH 7.4) were evaluated with Mota-Fab-VHH fusions. Albumus mice™ (HSA/hFcRn) which can mimic the physiological antibody clearance in humans were injected intraperitoneally (IP-single dose) with seven different test items in order to evaluate their clearance in this mouse model. The test items which were administered are described in detail below:
1. Motavizumab-Fab fragment was used as a control treatment condition. This specific molecule has the same basic structure with the test items of interest; however, its size is smaller (˜50 kDa).
2. Motavizumab-Fab with the irrelevant VHH 3Rab fused C-terminally to the light chain using a 20GS linker was used as a second control treatment condition. This specific molecule has similar size as the test items of interest (˜65 kDa); however, it does not have any affinity to albumin.
3. Motavizumab-Fab fragment with Alb23 fused C-terminally to the light chain using a 20GS linker. Alb23 is an anti-albumin VHH. This treatment condition was included as this molecule appears to have high affinity for albumin, but this affinity is pH independent.
4. Motavizumab-Fab fragment fused with 2H11 C-terminally to the light chain using a 20GS linker. Previous in vitro studies showed that this variant appears to have high affinity for albumin at pH 5.5, however this affinity is slightly reduced in pH 7.4, further indicating pH-dependent affinity.
5. Motavizumab-Fab fragment fused with 2H11 mutants v3, v8, v9 or v15 c-terminally to the light chain using a 20GS linker. After alanine scanning and in vitro characterization, these 2H11 mutants carry an alanine mutation in CDR3 (location 3, 8, 9 and 15, respectively) and appear to have reduced affinity at both pH 5.5 and pH 7.4 compared with the parental molecule 2H11 but a stronger pH dependent affinity.

Doses for the test items were 19 mg/kg (wt Fab) and 25 mg/kg (C-terminally fused Fab fragments) according to equimolar dosing of 30 mg/kg of Fc-ABDEG-Alb23 as reference which was used in previous studies.

For practical reasons, the experiment was divided into two parts that were performed consecutively:
Experiment 1: Treatment groups 1, 2, 3, 4
Experiment 2: Treatment groups 4, 5, 6, 7, 8

In total, forty-five (45) male and female Albumus mice™ were used for two experiments (treatment group 4 included in both studies). The Albumus mice™ at approximately 14-15 weeks of age were homozygous for the human FcRn transgene and human albumin transgene. The mice were tail marked for identification and housed individually in positively ventilated polysulfone cages with HEPA filtered air at a maximum density of up to 4 mice per cage. The normal temperature and relative humidity ranges in the animal rooms were 22+4° C. and 50±15%, respectively. Filtered tap water, acidified to a pH of 2.5 to 3.0, and standard lab chow were provided ad libitum.
1. Albumus mice™ were randomly distributed into 8 groups, 5 mice/group.
2. On Day −3 (−3d), mice were preloaded with IVIg.
3. On Day 0, at 0 hours, the test articles were administered IP to mice as follows:
   Mota-Fab at 19 mg/kg, n=5, Group 1
   Mota-Fab-LC-3Rab (Mota-Fab-3Rab) at 25 mg/kg, n=5, Group 2
   Mota-Fab-LC-Alb23 (Mota-Fab-Alb23) at 25 mg/kg, n=5, Group 3
   Mota-Fab-LC-2H11 (Mota-Fab-2H11) at 25 mg/kg, n=5, Group 4
   Mota-Fab-LC-2H11v3 (Mota-Fab-2H11v3) at 25 mg/kg, n=5, Group 5
   Mota-Fab-LC-2H11v8 (Mota-Fab-2H11v8) at 25 mg/kg, n=5, Group 6
   Mota-Fab-LC-2H11v9 (Mota-Fab-2H11v9) at 25 mg/kg, n=5, Group 7
   Mota-Fab-LC-2H11v15 (Mota-Fab-2H11v15) at 25 mg/kg, n=5, Group 8
4. 20 μL blood samples were collected from each mouse via tail vein according to the bleeding schedule shown in Table S9 below at 2 h, 1d, 2d, 3d, 4d, 7d, 8d.
5. Serum samples were assessed in the next ELISA-based assays (in the order of priority):
   a. Test items PK ELISA
   b. Human serum albumin ELISA

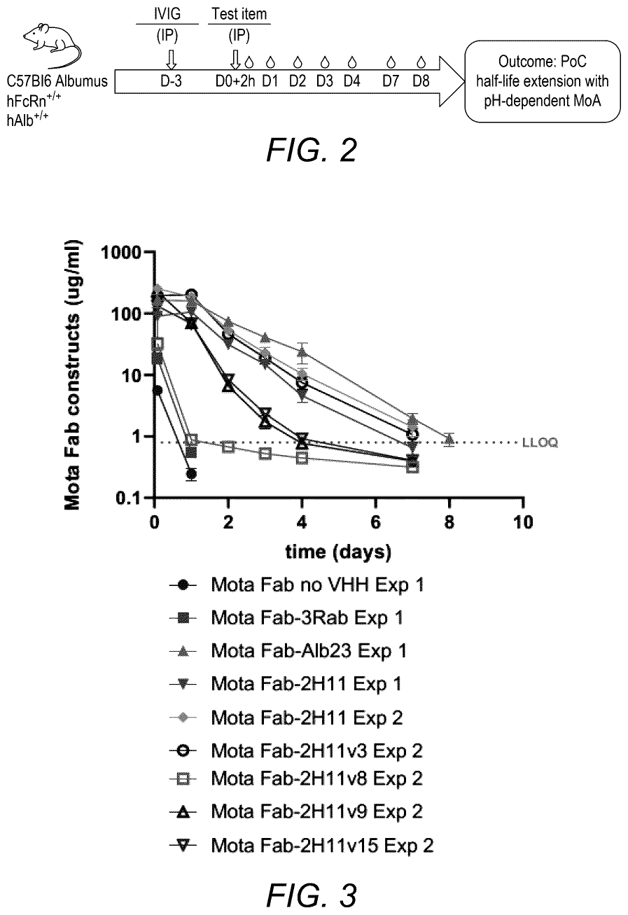

The protocol is also summarized in FIG. 2 and Table S9. Readouts on collected mouse serum were performed using PK, and HSA assays.

TABLE S9
In vivo protocol

| | | | | Conc. | | | | Volume test | Blood |
|---|---|---|---|---|---|---|---|---|---|
| | | | | test | | | | item | sampling |
| Groups | n | Dose | | item | | Frequency | Route | injection | points |
| Mota-Fab | 5 | 19 | mg/kg | 6.9 | mg/ml | SD | IP | 250 μl | D 0 + 2 h, D 1, |
| | | | | | | | | | D 2, D 3, D 4, |
| | | | | | | | | | D 7, D 8 |
| Mota-Fab-LC-3Rab | 5 | 25 | mg/kg | 7.81 | mg/ml | SD | IP | 250 μl | D 0 + 2 h, D 1, |
| (irrelevant VHH) | | | | | | | | | D 2, D 3, D 4, |
| | | | | | | | | | D 7, D 8 |
| Mota-Fab-LC-Alb23 | 5 | 25 | mg/kg | 9.9 | mg/ml | SD | IP | 250 μl | D 0 + 2 h, D 1, |
| (high affinity, no pH | | | | | | | | | D 2, D 3, D 4, |
| dependency) | | | | | | | | | D 7, D 8 |
| Mota-Fab-LC-2H11 | 5 | 25 | mg/kg | 9.53 | mg/ml | SD | IP | 250 μl | D 0 + 2 h, D 1, |
| (high affinity, pH | | | | | | | | | D 2, D 3, D 4, |
| dependency) | | | | | | | | | D 7, D 8 |
| Mota-Fab-LC-2H11v3 | 5 | 25 | mg/kg | 8.97 | mg/ml | SD | IP | 250 μl | D 0 + 2 h, D 1, |
| (low affinity, pH | | | | | | | | | D 2, D 3, D 4, |
| dependency) | | | | | | | | | D 7, D 8 |
| Mota-Fab-LC-2H11v3 | 5 | 25 | mg/kg | 9.27 | mg/ml | SD | IP | 250 μl | D 0 + 2 h, D 1, |
| (low affinity, pH | | | | | | | | | D 2, D 3, D 4, |
| dependency) | | | | | | | | | D 7, D 8 |
| Mota-Fab-LC-2H11v9 | 5 | 25 | mg/kg | 9.15 | mg/ml | SD | IP | 250 μl | D 0 + 2 h, D 1, |
| (low affinity, pH | | | | | | | | | D 2, D 3, D 4, |
| dependency) | | | | | | | | | D 7, D 8 |
| Mota-Fab-LC- | 5 | 25 | mg/kg | 9.48 | mg/ml | SD | IP | 250 μl | D 0 + 2 h, D 1, |

| | |
|---|---|
| 2H11v15 (low affinity, | D 2, D 3, D 4, |
| pH dependency) | D 7, D 8 |

Serum concentrations of the Mota-Fab-VHHs were plotted as an average per group over time during the course of the study and are shown in FIG. 3. The datapoints show the mean±SD of 5 animals per group.

Mota-Fab-VHHs that did not bind albumin, e.g., Mota-Fab (˜ 50 kDa) and Mota-Fab-3Rab (˜65 kDa), showed a relatively short half-life with serum concentrations undetectable after 1 day. This data shows that molecular weight is not the main driver of serum persistence. Mota-Fab-2H11, containing a reverse pH-dependent anti-albumin VHH, showed a comparable half-life with Mota-Fab-Alb23. Mota-Fab coupled to 2H11 alanine variants with lowered affinity to albumin at pH 7.4 (see Table S10 using Biacore 8K+, in single cycle kinetics (SCK) protocol. Human serum albumin proteins was immobilized on CM5 chips and the Fab-VHH variants were injected in-solution in a eight step 1.5-fold dilution series in 1×HBS-EP+pH 7.4 or citrate buffer pH5.5 during 2 minutes at 30 μl/min.) showed a serum half-life in correspondence with their albumin binding affinity. Mota-Fab-2H11-v9 and Mota-Fab-2H11-v15 showed reduced half-life compared to Mota fusions with higher affinity for HSA, however they still showed a prolonged PK profile. In contrast, Mota-Fab-2H11-v8 showed no detectable binding to albumin at both pH 5.5 and pH 7.4 in SPR (data not shown) and showed a similar half-life as Mota-Fab and Mota-Fab-3Rab.

TABLE S10
Binding kinetics properties and theoretical anti-albumin: Albumin complex formation

| | | | | Concentration | | | | | |
|---|---|---|---|---|---|---|---|---|---|
| | | | | Complexed | | | | | |
| | | | | Mota-FabVHH | | | | | |
| | | | | at day 1 | | | | | |
| | KD (nM) | | | (100 μg/ml) | | VHH Bound/unbound (%) | | | |
| Clone | pH 5.5 | pH 7.4 | ratio | pH 5.5 | pH 7.4 | pH 5.5 | | pH 7.4 | |
| Mota-Fab-Alb23 | 12.3 | 65 | 5.28 | 1538 | 1538 | 100.0 | 0.0 | 100.0 | 0.0 |
| Mota-Fab-2H11wt | 2.85 | 264 | 92.63 | 1538 | 1537 | 100.0 | 0.0 | 100.0 | 0.0 |
| Mota-Fab-2H11v3 | 94.9 | 2580 | 27.19 | 1538 | 1531 | 100.0 | 0.0 | 100.0 | 0.0 |
| Mota-Fab-2H11v9 | 21200 | 2.67 × 10⁶ | 126 | 1486 | 283 | 96.6 | 3.4 | 18.4 | 81.6 |
| Mota-Fab-2H11v15 | 130000 | 5.10 × 10⁷ | 392 | 1264 | 18 | 82.2 | 17.8 | 1.2 | 98.8 |

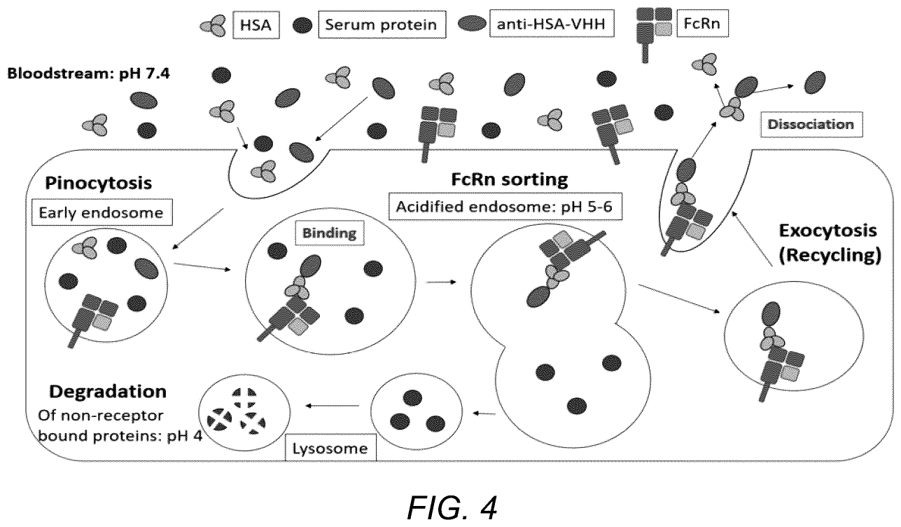

This suggests that Mota-Fab-2H11v9 and -2H11v15 have very low binding for HSA in serum and therefore only recycle through the binding to albumin in the endosome. As shown in FIG. 4, anti-HSA-VHH with reduced binding of HSA in the serum, but increased binding in the endosome tends to result in recycling of both HSA and the anti-HSA-VHH, without affecting the size of the anti-HSA-VHH. We have shown clones that bind HSA in a pH dependent manner that have desirable half-life and are expected to have better biodistribution in tissues and tumors upon administration.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. An antigen-binding molecule that specifically binds to HSA, wherein the equilibrium dissociation constant of the antigen-binding molecule to HSA at pH 5.5 and/or pH 6.0 is less than 0.140 mM and the equilibrium dissociation constant of the antigen-binding molecule to HSA at pH 7.4 is at least 2.4 Mm.

2. The antigen-binding molecule of claim 1, wherein the equilibrium dissociation constant of the antigen-binding molecule to HSA at pH 5.5 is less than 0.03 mM and the equilibrium dissociation constant of the antigen-binding molecule to HSA at pH 7.4 is at least 11.5 mM.

3. An antigen-binding molecule that specifically binds to HSA, wherein the binding affinity of the antigen-binding molecule to HSA at pH 5.5 and/or pH 6.0 is at least 100-fold higher than the binding affinity of the antigen-binding molecule to HSA at pH 7.4.

4. The antigen-binding molecule of claim 3, wherein the binding affinity is measured by surface plasmon resonance.

5. The antigen-binding molecule of claim 1, wherein the antigen-binding molecule is selected from a Fab fragment, a sdAb, an scFv, an antibody mimetic, HSA, or an antigen-binding fragment thereof.

6. The antigen-binding molecule of claim 5, wherein the sdAb is a VHH fragment.

7. The antigen-binding molecule of claim 6, further comprising one or more amino acids added to the C-terminal end of the VHH fragment.

8. The antigen-binding molecule of claim 7, wherein the one or more amino acids are selected from the group consisting of:
a) A;
b) AG;
c) GG; and
d) PP.

9. A fusion protein comprising the antigen-binding molecule of claim 1 and a therapeutic protein.

10. The fusion protein of claim 9, wherein the therapeutic protein is a small peptide therapeutic.

11. The fusion protein of claim 9, wherein the therapeutic protein is an antibody or fragment thereof.

12. The fusion protein of claim 9, wherein the antigen-binding molecule is fused to the therapeutic protein via a linker.

13. The fusion protein of claim 12, wherein the linker is a non-cleavable linker.

14. The fusion protein of claim 12, wherein the linker is a peptide linker.

15. The fusion protein of claim 14, wherein the peptide linker is a GS linker.

16. The fusion protein of claim 11, wherein the antigen-binding molecule is fused to the antibody or fragment thereof via an IgG hinge region or portion thereof.

17. The fusion protein of claim 9, wherein the therapeutic protein is an Fc region comprising a first Fc domain and a second Fc domain, and wherein the antigen-binding molecule is fused to the first Fc domain or the second Fc domain via an IgG hinge region or portion thereof.

18. An isolated polynucleotide or polynucleotides encoding the antigen-binding molecule of claim 1.

19. An expression vector comprising the isolated polynucleotide or polynucleotides of claim 18.

20. A host cell comprising the expression vector of claim 19.

21. A method for producing an antigen-binding molecule, comprising culturing the host cell of claim 20 under conditions which permit the expression of the antigen-binding molecule.

22. A pharmaceutical composition comprising an antigen-binding molecule of claim 1 and at least one pharmaceutically acceptable carrier.

23. (canceled)

24. A method of increasing serum half-life of a therapeutic protein comprising fusing an antigen-binding molecule of claim 1 to a therapeutic protein.

25.-26. (canceled)


SEQUENCE LISTING (195 sequences)

SEQ ID NO: 1 - protein, 15 aa, synthetic construct
EPKSCDKTHTCPPCP

SEQ ID NO: 2 - protein, 20 aa, synthetic construct
GGGGSGGGGSGGGGSGGGGS

SEQ ID NO: 3 - protein, 10 aa, synthetic construct
GGGGSGGGGS

SEQ ID NO: 4 - protein, 30 aa, synthetic construct
GGGGSGGGGSGGGGSGGGGSGGGGSGGGGS

SEQ ID NO: 5 - protein, 5 aa, synthetic construct
SNTMG

SEQ ID NO: 6 - protein, 17 aa, synthetic construct
AITWSGGTTYYADSVKG

SEQ ID NO: 7 - protein, 19 aa, synthetic construct
EGPKWEPWNGIYHPADFGS

SEQ ID NO: 8 - protein, 19 aa, synthetic construct
EGPKWEPWAGIYHPADFGS

SEQ ID NO: 9 - protein, 19 aa, synthetic construct
EGPKWEPANGIYHPADFGS

SEQ ID NO: 10 - protein, 19 aa, synthetic construct
EGPKWEPWNGIYHPAAFGS

SEQ ID NO: 11 - protein, 19 aa, synthetic construct
EGAKWEPWNGIYHPADFGS

SEQ ID NO: 12 - protein, 19 aa, synthetic construct
EGPKWEPWNGIAHPADFGS

SEQ ID NO: 13 - protein, 19 aa, synthetic construct
EGPKWEPWNGIYHPADAGS

SEQ ID NO: 14 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHPADFGSWGQGTQVTVSS

SEQ ID NO: 15 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWAGIYHPADFGSWGQGTQVTVSS

SEQ ID NO: 16 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPANGIYHPADFGSWGQGTQVTVSS

SEQ ID NO: 17 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHPAAFGSWGQGTQVTVSS

SEQ ID NO: 18 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGAKWEPWNGIYHPADFGSWGQGTQVTVSS

SEQ ID NO: 19 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIAHPADFGSWGQGTQVTVSS

SEQ ID NO: 20 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHPADAGSWGQGTQVTVSS

SEQ ID NO: 21 - protein, 19 aa, synthetic construct
AGPKWEPWNGIYHPADFGS

SEQ ID NO: 22 - protein, 19 aa, synthetic construct
EAPKWEPWNGIYHPADFGS

SEQ ID NO: 23 - protein, 19 aa, synthetic construct
EGPAWEPWNGIYHPADFGS

SEQ ID NO: 24 - protein, 19 aa, synthetic construct
EGPKAEPWNGIYHPADFGS

SEQ ID NO: 25 - protein, 19 aa, synthetic construct
EGPKWAPWNGIYHPADFGS

SEQ ID NO: 26 - protein, 19 aa, synthetic construct
EGPKWEAWNGIYHPADFGS

SEQ ID NO: 27 - protein, 19 aa, synthetic construct
EGPKWEPWNAIYHPADFGS

SEQ ID NO: 28 - protein, 19 aa, synthetic construct
EGPKWEPWNGAYHPADFGS

SEQ ID NO: 29 - protein, 19 aa, synthetic construct
EGPKWEPWNGIYAPADFGS

SEQ ID NO: 30 - protein, 19 aa, synthetic construct
EGPKWEPWNGIYHAADFGS

SEQ ID NO: 31 - protein, 19 aa, synthetic construct
EGPKWEPWNGIYHPADFAS

SEQ ID NO: 32 - protein, 19 aa, synthetic construct
EGPKWEPWNGIYHPADFGA

SEQ ID NO: 33 - protein, 5 aa, synthetic construct
AYPMG

SEQ ID NO: 34 - protein, 18 aa, synthetic construct
GISWFAGATTDYADSVEG

SEQ ID NO: 35 - protein, 18 aa, synthetic construct
SHQLPGGRPGRDMDSYSY

SEQ ID NO: 36 - protein, 18 aa, synthetic construct
AHQLPGGRPGRDMDSYSY

SEQ ID NO: 37 - protein, 18 aa, synthetic construct
SAQLPGGRPGRDMDSYSY

SEQ ID NO: 38 - protein, 18 aa, synthetic construct
SHALPGGRPGRDMDSYSY

SEQ ID NO: 39 - protein, 18 aa, synthetic construct
SHQAPGGRPGRDMDSYSY

SEQ ID NO: 40 - protein, 18 aa, synthetic construct
SHQLAGGRPGRDMDSYSY

SEQ ID NO: 41 - protein, 18 aa, synthetic construct
SHQLPAGRPGRDMDSYSY

SEQ ID NO: 42 - protein, 18 aa, synthetic construct
SHQLPGARPGRDMDSYSY

SEQ ID NO: 43 - protein, 18 aa, synthetic construct
SHQLPGGAPGRDMDSYSY

SEQ ID NO: 44 - protein, 18 aa, synthetic construct
SHQLPGGRAGRDMDSYSY

SEQ ID NO: 45 - protein, 18 aa, synthetic construct
SHQLPGGRPARDMDSYSY

SEQ ID NO: 46 - protein, 18 aa, synthetic construct
SHQLPGGRPGADMDSYSY

SEQ ID NO: 47 - protein, 18 aa, synthetic construct
SHQLPGGRPGRAMDSYSY

SEQ ID NO: 48 - protein, 18 aa, synthetic construct
SHQLPGGRPGRDADSYSY

SEQ ID NO: 49 - protein, 18 aa, synthetic construct
SHQLPGGRPGRDMASYSY

SEQ ID NO: 50 - protein, 18 aa, synthetic construct
SHQLPGGRPGRDMDAYSY

SEQ ID NO: 51 - protein, 18 aa, synthetic construct
SHQLPGGRPGRDMDSASY

SEQ ID NO: 52 - protein, 18 aa, synthetic construct
SHQLPGGRPGRDMDSYAY

SEQ ID NO: 53 - protein, 18 aa, synthetic construct
SHQLPGGRPGRDMDSYSA

SEQ ID NO: 54 - protein, 5 aa, synthetic construct
SYAMG

SEQ ID NO: 55 - protein, 17 aa, synthetic construct
TINWSGGSTYYADSVKG

SEQ ID NO: 56 - protein, 15 aa, synthetic construct
DLYGPGFVLSMEYDY

SEQ ID NO: 57 - protein, 17 aa, synthetic construct
VISWSGGSTYYADSVKG

SEQ ID NO: 58 - protein, 19 aa, synthetic construct
EGPKWEPYNGIYSPADFGS

SEQ ID NO: 59 - protein, 5 aa, synthetic construct
GYAMG

SEQ ID NO: 60 - protein, 18 aa, synthetic construct
AISWFAGDTTDYADSVKG

SEQ ID NO: 61 - protein, 15 aa, synthetic construct
SSARPMSGRSDEYHY

SEQ ID NO: 62 - protein, 5 aa, synthetic construct
SYVMA

SEQ ID NO: 63 - protein, 17 aa, synthetic construct
SINWSGGSTYYAHSVKG

SEQ ID NO: 64 - protein, 14 aa, synthetic construct
DLYGLGYEVMSYDY

SEQ ID NO: 65 - protein, 5 aa, synthetic construct
NYNAG

SEQ ID NO: 66 - protein, 17 aa, synthetic construct
AINWSGGTTYYADSVKG

SEQ ID NO: 67 - protein, 14 aa, synthetic construct
DPDWSILNPREYDY

SEQ ID NO: 68 - protein, 5 aa, synthetic construct
TYGMG

SEQ ID NO: 69 - protein, 16 aa, synthetic construct
AINWKGDRYYADSVKG

SEQ ID NO: 70 - protein, 15 aa, synthetic construct
DSDGSAFSRRLEYDY

SEQ ID NO: 71 - protein, 5 aa, synthetic construct
GYAMG

SEQ ID NO: 72 - protein, 18 aa, synthetic construct
AISWFAGDTTDYANSVKG

SEQ ID NO: 73 - protein, 15 aa, synthetic construct
SSARPLSSRSDEYHY

SEQ ID NO: 74 - protein, 5 aa, synthetic construct
HSNMA

SEQ ID NO: 75 - protein, 16 aa, synthetic construct
HIHWRGSTTYLDSVKG

SEQ ID NO: 76 - protein, 16 aa, synthetic construct
DSARRILVTVSTSYDY

SEQ ID NO: 77 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAAGPKWEPWNGIYHPADFGSWGQGTQVTVSS

SEQ ID NO: 78 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEAPKWEPWNGIYHPADFGSWGQGTQVTVSS

SEQ ID NO: 79 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPAWEPWNGIYHPADFGSWGQGTQVTVSS

SEQ ID NO: 80 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKAEPWNGIYHPADFGSWGQGTQVTVSS

SEQ ID NO: 81 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWAPWNGIYHPADFGSWGQGTQVTVSS

SEQ ID NO: 82 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEAWNGIYHPADFGSWGQGTQVTVSS

SEQ ID NO: 83 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNAIYHPADFGSWGQGTQVTVSS

SEQ ID NO: 84 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGAYHPADFGSWGQGTQVTVSS

SEQ ID NO: 85 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYAPADFGSWGQGTQVTVSS

SEQ ID NO: 86 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHAADFGSWGQGTQVTVSS

SEQ ID NO: 87 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHPADFASWGQGTQVTVSS

SEQ ID NO: 88 - protein, 128 aa, synthetic construct
ELQVVESGGGLVQAGGSLRLSCAASGRTFRSNTMGWFRQAPGKEREFVAAITWSGGTTYYADSVKGRFAISGDNAKNTVYLQMNSLKPEDTAVYYCAAEGPKWEPWNGIYHPADFGAWGQGTQVTVSS

SEQ ID NO: 89 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMDSYSYWGHGTQVTVSS

SEQ ID NO: 90 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAIAHQLPGGRPGRDMDSYSYWGHGTQVTVSS

SEQ ID NO: 91 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISAQLPGGRPGRDMDSYSYWGHGTQVTVSS

SEQ ID NO: 92 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHALPGGRPGRDMDSYSYWGHGTQVTVSS

SEQ ID NO: 93 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQAPGGRPGRDMDSYSYWGHGTQVTVSS

SEQ ID NO: 94 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLAGGRPGRDMDSYSYWGHGTQVTVSS

SEQ ID NO: 95 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPAGRPGRDMDSYSYWGHGTQVTVSS

SEQ ID NO: 96 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGARPGRDMDSYSYWGHGTQVTVSS

SEQ ID NO: 97 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGAPGRDMDSYSYWGHGTQVTVSS

SEQ ID NO: 98 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRAGRDMDSYSYWGHGTQVTVSS

SEQ ID NO: 99 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPARDMDSYSYWGHGTQVTVSS

SEQ ID NO: 100 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGADMDSYSYWGHGTQVTVSS

SEQ ID NO: 101 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRAMDSYSYWGHGTQVTVSS

SEQ ID NO: 102 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDADSYSYWGHGTQVTVSS

SEQ ID NO: 103 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMASYSYWGHGTQVTVSS

SEQ ID NO: 104 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMDAYSYWGHGTQVTVSS

SEQ ID NO: 105 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMDSASYWGHGTQVTVSS

SEQ ID NO: 106 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMDSYAYWGHGTQVTVSS

SEQ ID NO: 107 - protein, 128 aa, synthetic construct
QLQVVESGGGLVQAGGSLRLSCAASERTFVAYPMGWFRQAPEKEREFVAGISWFAGATTDYADSVEGRFTISRDNAKNTAYLQMNNLKPEDTAVYYCAISHQLPGGRPGRDMDSYSAWGHGTQVTVSS

SEQ ID NO: 108 - protein, 124 aa, synthetic construct
EVQLVESGGGLVQAGGSLRLSCAASGRTFSSYAMGWFRQAPGKEREFVATINWSGGSTYYADSVKGRFTISRDNAKNTVYLQMNSLKPEDTAVYYCAADLYGPGFVLSMEYDYWGQGTQVTVSS

SEQ ID NO: 109 - protein, 128 aa, synthetic construct
QLQLVESGGGLVQAGGSLRLSCAASGRTFSSYAMGWFRQAPGKEREFVAVISWSGGSTYYADSVKGRFTISGDNAKNTVYLQMNSLKPEDTAVYYCATEGPKWEPYNGIYSPADFGSWGQGTQVTVSS

SEQ ID NO: 110 - protein, 125 aa, synthetic construct
EVQLVESGGGLVQAGGSLRLSCTASRRAFIGYAMGWFRQAPGKEREFVAAISWFAGDTTDYADSVKGRFTISRDNAKNTVYLQMNGLKPEDTAVYVCAASSARPMSGRSDEYHYWGQGTQVTVSS

SEQ ID NO: 111 - protein, 123 aa, synthetic construct
QLQLVESGGGLVQAGGSLRLSCAASGRAISSYVMAWFRQAPGKEREFVTSINWSGGSTYYAHSVKGRFTISRDNAKNTVYLQMNTLKPEDTAVYYCAADLYGLGYEVMSYDYWGQGTQVTVSS

SEQ ID NO: 112 - protein, 123 aa, synthetic construct
QLQLVESGGGLVQTGGSLRLSCAASGRTFSNYNAGWFRQVPGKEPEFVAAINWSGGTTYYADSVKGRFTISRDNAKNTVYLQMNSLKPEDTAVYYCAADPDWSILNPREYDYWGQGTRVTVSS

SEQ ID NO: 113 - protein, 123 aa, synthetic construct
QLQLVESGGGLVQAGGSLRLSCVASGRSFSTYGMGWFRQAPGAEREFAAAINWKGDRYYADSVKGRFTISRDNAKNTVFLEMNSLKPEDTGVYYCAADSDGSAFSRRLEYDYWGQGTQVTVSS

SEQ ID NO: 114 - protein, 125 aa, synthetic construct
QLQVVESGGGLVQAGGSLRLSCTTSGRAFIGYAMGWFRQAPGKEREFVAAISWFAGDTTDYANSVKGRFTISRDNAKNTVYLQMNSLKPEDTAVYVCAASSARPLSSRSDEYHYWGQGTQVTVSS

SEQ ID NO: 115 - protein, 124 aa, synthetic construct
QVQLVESGGGLVQAGGSLRLSCAASERAFSHSNMAWFRQAPGKEREFVAHIHWRGSTTYLDSVKGRFTISRDNAKNTVYLQMNGLRLEDTAVYYCAADSARRILVTVSTSYDYWGQGTQVTVSS

SEQ ID NO: 116 - protein, 5 aa, synthetic construct
SYWMY

SEQ ID NO: 117 - protein, 17 aa, synthetic construct
VINTSGGSTYYADSVKG

SEQ ID NO: 118 - protein, 11 aa, synthetic construct
GGLVVAGGLGS

SEQ ID NO: 119 - protein, 5 aa, synthetic construct
MYWMY

SEQ ID NO: 120 - protein, 17 aa, synthetic construct
AIDTRGGSTYYADSVKG

SEQ ID NO: 121 - protein, 11 aa, synthetic construct
GGLVRVGGLGS

SEQ ID NO: 122 - protein, 5 aa, synthetic construct
YYWMY

SEQ ID NO: 123 - protein, 5 aa, synthetic construct
HYYMN

SEQ ID NO: 124 - protein, 17 aa, synthetic construct
GINTGGGTTYYADSVKG

SEQ ID NO: 125 - protein, 11 aa, synthetic construct
GGIVAVGGFGS

SEQ ID NO: 126 - protein, 5 aa, synthetic construct
RYWIH

SEQ ID NO: 127 - protein, 17 aa, synthetic construct
GISIGDGTTYYADSVRG

SEQ ID NO: 128 - protein, 11 aa, synthetic construct
GGIVYFGAYDS

SEQ ID NO: 129 - protein, 5 aa, synthetic construct
DFVMH

SEQ ID NO: 130 - protein, 17 aa, synthetic construct
AIRWDDGSTYYAESMKG

SEQ ID NO: 131 - protein, 8 aa, synthetic construct
SVSASSNY

SEQ ID NO: 132 - protein, 5 aa, synthetic construct
RYWMH

SEQ ID NO: 133 - protein, 17 aa, synthetic construct
GINTGTGSTAYADSVKG

SEQ ID NO: 134 - protein, 11 aa, synthetic construct
GGLVVGGGYSD

SEQ ID NO: 135 - protein, 5 aa, synthetic construct
RYWIH

SEQ ID NO: 136 - protein, 17 aa, synthetic construct
GISIGDGTTYYADSVRG

SEQ ID NO: 137 - protein, 11 aa, synthetic construct
GGIVYFGAYDS

SEQ ID NO: 138 - protein, 13 aa, synthetic construct
TGSSSNIGDNYVN

SEQ ID NO: 139 - protein, 7 aa, synthetic construct
DNTKRAS

SEQ ID NO: 140 - protein, 11 aa, synthetic construct
SSWDDSLSGLV

SEQ ID NO: 141 - protein, 11 aa, synthetic construct
GGDNIGSKNAH

SEQ ID NO: 142 - protein, 7 aa, synthetic construct
KDSNRPS

SEQ ID NO: 143 - protein, 11 aa, synthetic construct
QVWDSSANAAV

SEQ ID NO: 144 - protein, 14 aa, synthetic construct
AGTSSDVGYGNYVS

SEQ ID NO: 145 - protein, 7 aa, synthetic construct
DVSKRAS

SEQ ID NO: 146 - protein, 9 aa, synthetic construct
ASYRSGTTV

SEQ ID NO: 147 - protein, 17 aa, synthetic construct
KSSQSVVSGSNQKSLLN

SEQ ID NO: 148 - protein, 7 aa, synthetic construct
LASTRES

SEQ ID NO: 149 - protein, 9 aa, synthetic construct
QQGYGTPFS

SEQ ID NO: 150 - protein, 11 aa, synthetic construct
QASQNIISYLA

SEQ ID NO: 151 - protein, 7 aa, synthetic construct
GASRLQT

SEQ ID NO: 152 - protein, 9 aa, synthetic construct
LQHLTYPLT

SEQ ID NO: 153 - protein, 17 aa, synthetic construct
KSSRSVLYSSNQKNYLA

SEQ ID NO: 154 - protein, 9 aa, synthetic construct
QQGYDVPVT

SEQ ID NO: 155 - protein, 11 aa, synthetic construct
QASQSISSYLA

SEQ ID NO: 156 - protein, 8 aa, synthetic construct
LQQKLPVT

SEQ ID NO: 157 - protein, 120 aa, synthetic construct
EVQLVESGGGLVQPGGSLRLSCAASGFTFSSYWMYWVRQAPGKGLEWVSVINTSGGSTYYADSVKGRFTISRDNAKNTLYLQMNNLKSEDTAVYYCAKGGLVVAGGLGSWGQGTQVTVSS

SEQ ID NO: 158 - protein, 110 aa, synthetic construct
QPVLNQPPSVSGSPGQKFTISCTGSSSNIGDNYVNWYQHLPGTAPKLLIYDNTKRASGVPDRFSGSKSGTSASLTITELQAEDEADYYCSSWDDSLSGLVFGGGTRLTVL

SEQ ID NO: 159 - protein, 120 aa, synthetic construct
EVQLQESGGGLVQPGGSLRLSCAASGFTFSMYWMYWVRQAPGKGLEWVSAIDTRGGSTYYADSVKGRFTISRDNAKNTLYLQMNSLKPEDTALYYCATGGLVRVGGLGSWGQGTQVTVSS

SEQ ID NO: 160 - protein, 108 aa, synthetic construct
SYELTQSPSVSVALRQTAKITCGGDNIGSKNAHWYQQKPGQAPVLVIYKDSNRPSGIPERFSGSNSGNTATLTVSGAQAEDEADYYCQVWDSSANAAVFGGGTHLTVL

SEQ ID NO: 161 - protein, 120 aa, synthetic construct
QVQLVESGGGLVQPGGSLRLSCAASGFTFSYYWMYWVRQAPGKGLEWVSAIDTRGGSTYYADSVKGRFTISRDNAKNTLYLQMNSLKPEDTALYYCARGGLVRVGGLGSWGQGTQVTVSS

SEQ ID NO: 162 - protein, 109 aa, synthetic construct
QAVLTQPPSVSGSPGKTVTISCAGTSSDVGYGNYVSWYQQLPGMAPKLLIYDVSKRASGITDRFSASKSGNTASLTISGLQSEDEADYYCASYRSGTTVFGGGTHLTVL

SEQ ID NO: 163 - protein, 120 aa, synthetic construct
ELQLVESGGGLVQPGGSLRVSCAASGFTFSHYYMNWVRQAPGKGLEWVSGINTGGGTTYYADSVKGRFTISRDNAKNTLYLQMNSLKPEDTALYYCAKGGIVAVGGFGSWGQGTQVTVSS

SEQ ID NO: 164 - protein, 108 aa, synthetic construct; identical to SEQ ID NO: 160

SEQ ID NO: 165 - protein, 120 aa, synthetic construct
EVQLQESGGGLVQPGGSLRLSCAASGFTFGRYWIHWVRQAPGKGLEWVSGISIGDGTTYYADSVRGRFTISRDKAKNTLALTMNSLKSEDTAVYYCAKGGIVYFGAYDSWGQGTQVTVSS

SEQ ID NO: 166 - protein, 113 aa, synthetic construct
DIVMTQSPRSVTASVGEKVTINCKSSQSVVSGSNQKSLLNWYQQRPGQSPRLLIYLASTRESGVPDRFSGSGSTTDFTLTISSFQPEDVAVYYCQQGYGTPFSFGSGTRLEIK

SEQ ID NO: 167 - protein, 117 aa, synthetic construct
ELQLVESGGGLVQPGGSLRLSCAASGFTSDDFVMHWVRQAPGKGLEWVSAIRWDDGSTYYAESMKGRFTISRDNAKNTLYLQMNSLKSEDTAVYYCAKSVSASSNYWGQGTQVTVSS

SEQ ID NO: 168 - protein, 107 aa, synthetic construct
DIVMTQSPSSLSVSLGDRVTITCQASQNIISYLAWYQQKPGQTPKLLIYGASRLQTGVPSRFSGSGSGTSFTLTISGVEAEDVATYYCLQHLTYPLTFGQGTKVELK

SEQ ID NO: 169 - protein, 120 aa, synthetic construct
ELQLVESGGGLVQPGGSLRLACAASGFTFSRYWMHWVRQAPGKGLEWVSGINTGTGSTAYADSVKGRFTISRDNGKNTLYLQMNSLKSDDTAVYYCAKGGLVVGGGYSDWGQGTQVTVSS

SEQ ID NO: 170 - protein, 113 aa, synthetic construct
DIVMTQSPSSVTASAGEKVTINCKSSRSVLYSSNQKNYLAWYQQRPGQSPRLLIYLASTRESGVPDRFSGSGSTTDFTLTISSFQPEDVAVYYCQQGYDVPVTFGQGTKVELK

SEQ ID NO: 171 - protein, 120 aa, synthetic construct
QVQVQESGGGLVQPGGSLRLSCAASGFTFGRYWIHWVRQAPGKGLEWVSGISIGDGTTYYADSVRGRFTISRDKAKNTLALTMNSLKSEDTAVYYCAKGGIVYFGAYDSWGQGTQVTVSS

SEQ ID NO: 172 - protein, 106 aa, synthetic construct
DIVMTQSPSSLSASLGDRVTITCQASQSISSYLAWYQQKPGQAPKLLIYGASRLQTGVPSRFSGGGSGTSFTLTISGVEAEDVATYYCLQQKLPVTFGQGTKVELK

SEQ ID NO: 173 - protein, 5 aa, synthetic construct
SFGMS

SEQ ID NO: 174 - protein, 17 aa, synthetic construct
SISGSGSDTLYADSVKG

SEQ ID NO: 175 - protein, 6 aa, synthetic construct
GGSLSR

SEQ ID NO: 176 - protein, 115 aa, synthetic construct [sequence omitted]

SEQ ID NO: 177 - protein, 5 aa, synthetic construct
AAPMG

SEQ ID NO: 178 - protein, 5 aa, synthetic construct
AYAMG

SEQ ID NO: 179 - protein, 18 aa, synthetic construct
GASWFAGATTDYADSVEG

SEQ ID NO: 180 - protein, 18 aa, synthetic construct
GIAWFAGATTDYADSVEG

SEQ ID NO: 181 - protein, 18 aa, synthetic construct
GISAFAGATTDYADSVEG

SEQ ID NO: 182 - protein, 18 aa, synthetic construct
GISWAAGATTDYADSVEG

SEQ ID NO: 183 - protein, 18 aa, synthetic construct
GISWFAAATTDYADSVEG

SEQ ID NO: 184 - protein, 18 aa, synthetic construct
GISWFAGATADYADSVEG

SEQ ID NO: 185 - protein, 18 aa, synthetic construct
GISWFAGATTDAADSVEG

SEQ ID NO: 186 - protein, 128 aa, synthetic construct
QLQVVESGGGLVQAGGSLRLSCAASERTFVAAPMGWFRQAPEKEREFVAGISWFAGATTDYADSVEGRFTISRDNAKNTAYLQMNNLKPEDTAVYYCAISHQLPGGRPGRDMDSYSYWGHGTQVTVSS

SEQ ID NO: 187 - protein, 128 aa, synthetic construct
QLQVVESGGGLVQAGGSLRLSCAASERTFVAYAMGWFRQAPEKEREFVAGISWFAGATTDYADSVEGRFTISRDNAKNTAYLQMNNLKPEDTAVYYCAISHQLPGGRPGRDMDSYSYWGHGTQVTVSS

SEQ ID NO: 188 - protein, 128 aa, synthetic construct
QLQVVESGGGLVQAGGSLRLSCAASERTFVAYPMGWFRQAPEKEREFVAGASWFAGATTDYADSVEGRFTISRDNAKNTAYLQMNNLKPEDTAVYYCAISHQLPGGRPGRDMDSYSYWGHGTQVTVSS

SEQ ID NO: 189 - protein, 128 aa, synthetic construct
QLQVVESGGGLVQAGGSLRLSCAASERTFVAYPMGWFRQAPEKEREFVAGIAWFAGATTDYADSVEGRFTISRDNAKNTAYLQMNNLKPEDTAVYYCAISHQLPGGRPGRDMDSYSYWGHGTQVTVSS

SEQ ID NO: 190 - protein, 128 aa, synthetic construct
QLQVVESGGGLVQAGGSLRLSCAASERTFVAYPMGWFRQAPEKEREFVAGISAFAGATTDYADSVEGRFTISRDNAKNTAYLQMNNLKPEDTAVYYCAISHQLPGGRPGRDMDSYSYWGHGTQVTVSS

SEQ ID NO: 191 - protein, 128 aa, synthetic construct
QLQVVESGGGLVQAGGSLRLSCAASERTFVAYPMGWFRQAPEKEREFVAGISWAAGATTDYADSVEGRFTISRDNAKNTAYLQMNNLKPEDTAVYYCAISHQLPGGRPGRDMDSYSYWGHGTQVTVSS

SEQ ID NO: 192 - protein, 128 aa, synthetic construct
QLQVVESGGGLVQAGGSLRLSCAASERTFVAYPMGWFRQAPEKEREFVAGISWFAAATTDYADSVEGRFTISRDNAKNTAYLQMNNLKPEDTAVYYCAISHQLPGGRPGRDMDSYSYWGHGTQVTVSS

SEQ ID NO: 193 - protein, 128 aa, synthetic construct
QLQVVESGGGLVQAGGSLRLSCAASERTFVAYPMGWFRQAPEKEREFVAGISWFAGATADYADSVEGRFTISRDNAKNTAYLQMNNLKPEDTAVYYCAISHQLPGGRPGRDMDSYSYWGHGTQVTVSS

SEQ ID NO: 194 - protein, 128 aa, synthetic construct
QLQVVESGGGLVQAGGSLRLSCAASERTFVAYPMGWFRQAPEKEREFVAGISWFAGATTDAADSVEGRFTISRDNAKNTAYLQMNNLKPEDTAVYYCAISHQLPGGRPGRDMDSYSYWGHGTQVTVSS

SEQ ID NO: 195 - protein, 4 aa, synthetic construct
VTSS